(19) United States
(12) Patent Application Publication
(10) Pub. No.: US 2023/0263783 A1
(43) Pub. Date: Aug. 24, 2023

(54) CANCER TREATMENT BY COMBINED INHIBITION OF POLO-LIKE KINASE AND MICROTUBULE POLYMERIZATION

(71) Applicant: Massachusetts Institute of Technology, Cambridge, MA (US)

(72) Inventors: Michael B. Yaffe, West Roxbury, MA (US); Brian A. Joughin, Cambridge, MA (US); Jesse C. Patterson, Natick, MA (US)

(21) Appl. No.: 18/065,348

(22) Filed: Dec. 13, 2022

Related U.S. Application Data
(60) Provisional application No. 63/311,491, filed on Feb. 18, 2022.

(51) Int. Cl. A61K 31/437; A61K 31/40; A61K 31/5365; A61K 51/04; A61K 31/5025; A61K 9/00; A61P 35/00

(52) U.S. Cl. CPC A61K 31/437; A61K 31/40; A61K 31/5365; A61K 51/0463; A61K 31/5025; A61K 9/0019; A61K 9/0056; A61P 35/00

(57) ABSTRACT

An effective amount of one or more microtubule polymerization inhibitors is administered in combination with one or more polo-like kinase (Plk) inhibitors for treating cancer. Administration of the combination of the active agents can be effective to reduce cancer cell proliferation or viability in a subject with cancer to the same degree, or a greater degree, than administering to the subject the same amount of either active agent alone. The active agents can be administered together or separately. Methods of selecting and treating subjects with cancers are also provided.

### CROSS-REFERENCE TO RELATED APPLICATIONS

This application claims the benefit of and priority to U.S. Ser. No. 63/311,491, filed Feb. 18, 2022, which is specifically incorporated by reference herein in its entirety.

### STATEMENT REGARDING FEDERALLY SPONSORED RESEARCH

This invention was made with Government support under Grant No. R35 ES028374 and U54 CA217377 awarded by the National Institutes of Health. The Government has certain rights in the invention.

### FIELD OF THE INVENTION

The invention is generally in the field of combination therapies including a microtubule polymerization inhibitor and a polo-like kinase inhibitor for the treatment of cancer.

### BACKGROUND OF THE INVENTION

According to the American Cancer Society's global cancer facts report in 2009, the average 5-year survival rates for common pediatric and adult cancer subtypes in the North America are 65-95% and 14-56% respectively, which are still quite low (Gatta G, et al., Eur J Cancer. 2009(45):992-1005). The death rate from cancer in the United States has continued to decline. From 1991 to 2018, the cancer death rate has fallen 31%. This includes a 2.4% decline from 2017 to 2018—a record for the largest one-year drop in the cancer death rate.

Cancer remains the leading cause of childhood deaths in the United States, and the economic and financial burden for cancer research is increasing (AACR Releases AACR Cancer Progress Report 2015 American Association for Cancer Research 2015). Age-standard cancer mortality rates for all types of malignancies have decreased, but newer drugs only contribute to a small percentage of this improvement (Garattini S. Ann Oncol. 2003(14):813-6). This is primarily because the development of new pharmaceutical anti-cancer agents is laborious and expensive, requiring initial in vitro and in vivo experimentation, and subsequent clinical trials before receiving FDA approval. It is estimated that a newly designed drug takes 15 years to enter the pharmaceutical market (DiMasi JA, et al., J Health Econ. 2003(22):151-85). Consequently, it is important to find more efficient methodical approaches that are also economically feasible. Newer approaches that do not rely solely on a single agent's traditional cytotoxicity profile are required in order to provide a more targeted, efficient and enhanced form of cancer therapy.

It is an object of the invention to provide compositions and methods for use thereof for treatment of cancers.

It is another object of the invention to provide compositions and methods for treating cancers associated with overexpression of polo-like kinase 1 (Plk1).

It is yet another object of the invention to provide compositions and methods for treating cancers with little or no systemic toxicity.

### SUMMARY OF THE INVENTION

One or more microtubule targeting agents or microtubule polymerization inhibitors (referred to jointly as microtubule polymerization inhibitors) is administered in combination with one or more polo-like kinase (Plk) inhibitors for treating cancer. The combination of the active agents can be effective to reduce cancer cell proliferation or viability in a subject with cancer to a greater degree than administering to the subject the same amount of either active agent alone or the expected degree of efficacy for the combination. Both classes of drugs function by blocking the process of mitosis, and therefore the effects of combining both drugs to treat cancer would be expected, by one skilled in the art, to be, at best, only additive. Studies showed unexpected results for the specific combination of Plk1 inhibitors with microtubule polymerization inhibitors, with efficacy being statistically significantly more than additive. This is in contrast to results obtained using combinations with other anti-mitotic drugs, such as Aurora kinase inhibitors and anti-microtubule drugs.

Microtubule polymerization inhibitors, such as maytansinoids, nocodazole, vincristine, and TH588, in combination with a Plk inhibitor, such as BI2536, BI6727 (volasertib), GSK461364, and onvansertib, have shown a reduction in the viability and proliferation of cancer cells. The combination therapies can be used to improve the efficacy of one or the other of the active agents, or to re-sensitize cells that have become resistant to a dose (e.g., the maximum dose) of one or the other active agents when it is administered alone. Examples show the combination displayed more than additive efficacy relative to the effect of each agent administered alone in reducing cancer cell proliferation or viability in a subject with cancer.

Pharmaceutical compositions including an effective amount of a combination of a microtubule polymerization inhibitor and a Plk inhibitor can be administered together or separately. Methods of selecting and treating subjects with cancers are also provided. Typically, administration of the combination of the two active agents (i.e., microtubule polymerization inhibitor and Plk inhibitor) is effective to reduce cancer cell proliferation or viability in a subject with cancer to a greater degree than administering to the subject the same amount of the microtubule polymerization inhibitor alone or the same amount of the Plk inhibitor alone. In the most preferred embodiments, the reduction in cancer cell proliferation or viability in the subject with cancer is more than the additive reduction achieved by administering the microtubule polymerization inhibitor alone or the Plk inhibitor alone. In some subjects with tumors, the combination is effective to reduce tumor burden, reduce tumor progression, reduce the rate of tumor cell proliferation, or a combination thereof.

Preferably, the microtubule polymerization inhibitor binds to a site on tubulin such as the laulimalide-, taxane/epothilone-, vinca alkaloid-, or colchicine-binding sites. Exemplary vinca alkaloids include vincristine, vinblastine, vinorelbine, vindesine, and vinflunine. In one embodiment, the microtubule polymerization inhibitor is vincristine, or a prodrug, analog, or derivative, or pharmaceutically acceptable salt thereof. Exemplary microtubule polymerization inhibitors that bind to the colchicine-binding site on tubulin include nocodazole, TH588, colchicinoids, combretastatins, ombrabulin, phenstatin, podophyllotoxin, steganacin, curacin A, 2-methoxyestradiol, ABT-751, T138067, BNC-105P, indibulin, EPC2407, MPI-0441138, MPC-6827, CYT997, MN-029, CI-980, CP248, CP461, and TN16. In some embodiments, the microtubule polymerization inhibitors are monomethyl auristatin E, monomethyl auristatin F, and maytansinoids such as DM1 and DM4. In preferred embodiments, the microtubule polymerization inhibitors are conjugated via a linker to an antibody or an antigen binding fragment thereof that specifically binds to a cell surface molecule highly expressed in a tumor cell compared to healthy cells, for example, CD33, CD30, HER2, CD22, CD79b, Nectin4, trophoblast cell surface antigen (TROP-2), BCMA, and CD19. In some embodiments, the antibody-drug conjugates are one or more of ADCETRIS® (brentuximab vedotin), KADCYLA® (ado-trastuzumab emtansine), POLIVY® (polatuzumab vedotin-piiq), PADCEV® (enfortumab vedotin-ejfv), BLENREP® (belantamab mafodotin-blmf), and TIVDAK® (tisotumab vedotin-tftv).

Typically, the class of Plk inhibitors include dihydropteridinones, pyridopyrimidines, aminopyrimidines, substituted thiazolidinones, pteridine derivatives, dihydroimidazo[1,5-f]pteridines, metasubstituted thiazolidinones, benzyl styryl sulfone analogues, stilbene derivatives, 4,5-dihydro-1H-pyrazolo[4,3-h]quinazoline derivatives, and combinations thereof. In some embodiments, the Plk inhibitors are onvansertib, BI2536, volasertib (BI6727), GSK461364, HMN-176, HMN-214, rigosertib (ON-01910), MLN0905, TKM-080301, TAK-960, NMS-1286937, Ro3280 or CYC140. In preferred embodiments, the Plk1 inhibitor is onvansertib, or an analogue, derivative, or prodrug thereof.

Methods for treating cancer in a subject in need thereof include administering to the subject an effective amount of a composition for reducing or inhibiting the quantity and/or activity of microtubule polymerization and Plk signaling in cancer cells in the subject to reduce cancer cell proliferation and/or reduce cancer cell viability in the subject.
Typically, the amount of the composition does not reduce the proliferation and/or viability of healthy cells in the subject. In preferred embodiments, the methods administer an effective amount of a microtubule polymerization inhibitor and a Plk inhibitor. Preferably, the composition does not reduce or minimally reduce the proliferation and/or viability of healthy cells in the subject.

Typically, the composition is administered to the subject by a route such as intravenous, intramuscular, intravascular, intrapericardial, intrathecal, intracapsular, intraorbital, intracardiac, intraperitoneal, subcutaneous, intraarticular, subarachnoid, intraspinal, and oral. In some embodiments, the microtubule polymerization inhibitor and the Plk inhibitor are administered separately and independently at the same time or at different times. In some embodiments, the microtubule polymerization inhibitor is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the Plk inhibitor to the subject. In other cases, the Plk inhibitor is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the microtubule polymerization inhibitor to the subject.

Generally, the methods using the combination treatment are effective to treat cancer in a human. Exemplary cancer types include prostate cancer, breast cancer, ovarian cancer, colorectal cancer, pancreatic cancer, head and neck cancer, bladder cancer, and acute myeloid leukemia. In one embodiment, the cancer is castrate resistant prostate cancer. In some embodiments, the cancer that is sensitive to the methods is characterized by reduced expression or down-regulation of one or more genes or gene products involved in the mitotic spindle or mitotic spindle assembly. In some embodiments, the cancer is characterized by overexpression of Plk1. In some instances, the cancer cells are insensitive to microtubule polymerization inhibitor when microtubule polymerization inhibitor is administered without co-administration of the Plk inhibitor. In some embodiments, the methods include one or more additional therapies or procedures such as surgery or radiation therapy, administering one or more immune checkpoint modulators such as PD-1 antagonists, PD-1 ligand antagonists, and CTLA4 antagonists, or adoptive T cell therapy, and/or a cancer vaccine.

Kits include one or more microtubule polymerization inhibitors and one or more polo-like kinase (Plk) inhibitors in an amount effective to reduce cancer cell proliferation and/or reduce cancer cell viability in the subject, and instructions for use according to the described methods.

### BRIEF DESCRIPTION OF THE DRAWINGS

FIGS. 1A-1C are graphs showing the level of viability, mitotic arrest marker, and apoptotic marker achieved by the combination of a Plk1 inhibitor and a microtubule polymerization inhibitor is more than additive of the results achieved by the individual components alone. FIG. 1A shows percent (%) viability of C4-2 castrate resistant prostate cancer (CRPC) cells treated with increasing concentrations of TH588 (in μM) in the absence (black line; [symbol] ) or presence (grey line; [symbol] ) of the indicated amount of a Plk1 inhibitor, BI2536 for five days. The expected viability according to the Bliss Independence model of drug additivity was calculated (dashed black line; [symbol] ). Mean±SEM for three experiments are shown. FIG. 1B shows percent (%) of C4-2 CRPC cells that stained positive for phosphorylation of serine 10 on Histone H3, a marker for mitosis, from 0 to 48 hours post addition of each of vehicle control (DMSO; black line; [symbol] ), Plk1 inhibitor (B1I2536, light grey line; [symbol] ), a microtubule polymerization inhibitor (TH588, dark grey line; [symbol] ), or the combination (dashed black line; “ [symbol] ”). After the indicated amount of time, cells were collected, fixed, stained with DAPI and an anti-phospho-Ser10 Histone H3 antibody, and then analyzed by flow cytometry. Mean±SEM for three experiments are shown. FIG. 1C shows percent (%) of C4-2 CRPC that stained positive for cleaved caspase-3, a marker for apoptotic cell death, from 0 to 48 hours post addition of each of vehicle control (DMSO; black bar), Plk1 inhibitor (B1I2536, light grey bar), a microtubule polymerization inhibitor (TH588, dark grey bar), and the combination (dashed bar). After the indicated amount of time, cells were collected, fixed, stained with an anti-cleaved caspase-3 antibody, and then analyzed by flow cytometry. Mean±SEM for three experiments are shown.

FIG. 2A is a schematic showing a tumor-implantable device for multiplexed drug delivery in vivo in xenograft tumors. Microwells in this device are loaded with distinct drugs or drug combinations. Upon implantation into a tumor, drugs are solvated and diffuse into the surrounding tumor creating spatially distinct drug concentration gradients. The tumor tissue surrounding the device is the collected, fixed, sectioned, and stained using antibodies to detect apoptotic cancer cell death. FIG. 2B is a bar graph showing percentage of cells positive for the apoptotic marker cleaved caspase-3 within a 400-μm radius of the wells containing B12536, TH588, or half-dose B12536 in combination with half-dose TH588. Bars indicate the mean of measurements in three tumors±SEM, ** p<0.01 using a two-tailed Student's t-test.

FIG. 3 is a swarm plot showing the degree of more than additive effect (Bliss Volume) observed in individual cell lines separated by tissue of origin and transformation status. Above the dashed line are cancer cell lines from the indicated tissues of origin, below the dashed line are non-cancer cell lines.

FIG. 4 is a schematic showing the components of an antibody-drug conjugate (ADC) having three components: the antibody having binding specificity towards an epitope that is enriched on the surface of cancer cells relative to normal cells, the cytotoxic agent, and a chemical linker that connects them.

FIGS. 5A and 5B are line graphs showing percent viability (0-120%) of C4-2 CRPC cells as a function of concentrations (in nM) of the maytansinoids DM1 (FIG. 5A) or DM4 (FIG. 5B) in the absence (black line, [symbol] ) or presence (grey line; [symbol] ) of the indicated amount of a Plk1 inhibitor, onvansertib. Viability was assessed after five days. The expected response (dashed black line; [symbol] ) was calculated according to the Bliss Independence model of drug additivity.

FIGS. 6A-6N are line graphs presenting data from a panel of fourteen ovarian cancer cell lines treated with increasing concentrations of the maytansinoid DM4 in the absence (black line) or presence (grey line) of the indicated amount of the Plk1 inhibitor onvansertib for five days, including CAOV3 (FIG. 6A), OVCAR4 (FIG. 6B), COV362 (FIG. 6C), OVCAR8 (FIG. 6D), TOV21G (FIG. 6E), OV90 (FIG. 6F), TOV112D (FIG. 6G), 59M (FIG. 6H), OAW28 (FIG. 6I), OAW42 (FIG. 6J), JHOS2 (FIG. 6K), JHOS4 (FIG. 6L), SKOV-3 (FIG. 6M), and ES-2 (FIG. 6N). Cells were treated with a dose matrix of these drugs and the response matrix was fit to sigmoid curves. The response to each drug concentration was fit independently but constrained by orthogonal sigmoids. Doses of onvansertib shown were chosen based on a 20-30% reduction in viability we used in isolation. Individual data points from the experiment in triplicate are shown as black and grey circles for the absence or presence of onvansertib, respectively. The expected response (dashed line) was calculated from the sigmoid fits using the Bliss independence model of drug additivity.

FIGS. 7A-7D are line graphs presenting data from a panel of four bladder cancer cell lines treated with increasing concentrations of the microtubule polymerization inhibitor monomethyl auristatin E (MMAE) in the absence (black line) or presence (grey line) of the indicated amount of the Plk1 inhibitor onvansertib for five days, including HT1197 (FIG. 7A), HT1376 (FIG. 7B), J82 (FIG. 7C), and UMUC3 (FIG. 7D). Data were analyzed and presented similarly to those in FIGS. 6A-6N.

FIGS. 8A-8F are line graphs presenting data from a panel of six HER2+ breast cancer cell lines treated with increasing concentrations of the maytansinoid DM1 in the absence (black line) or presence (grey line) of the indicated amount of the Plk1 inhibitor onvansertib for five days, including AU565 (FIG. 8A), HCC1419 (FIG. 8B), HCC1569 (FIG. 8C), HCC1954 (FIG. 8D), JIMT1 (FIG. 8E), and ZR7530 (FIG. 8F). Data were analyzed and presented similarly to those in FIGS. 6A-6N.

### DETAILED DESCRIPTION OF THE INVENTION

### I. Definitions

The term “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. The term encompasses not only intact polyclonal or monoclonal antibodies, but also fragments thereof (such as Fab, Fab′, F(ab′)2, Fv), single chain (ScFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody refers to one or more fragments of an intact antibody that retain the ability to specifically bind to a given antigen (e.g., HER2, Trop-2). Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CHi domains; an Fv fragment consisting of the VL and VH domains of a single arm of an antibody; a single domain antibody (dAb) fragment and an isolated complementarity determining region (CDR).

An antibody, an antibody conjugate, or a polypeptide that “preferentially binds” or “specifically binds” (used interchangeably herein) to a target (e.g., HER2 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a HER2 epitope is an antibody that binds this epitope with greater affinity, avidity, more readily, and/or with greater duration than it binds to other HER2 epitopes or non-HER2 epitopes. It is also understood that an antibody (or moiety or epitope) that specifically or preferentially binds to a first target may or may not specifically or preferentially bind to a second target. As such, “specific binding” or “preferential binding” does not require exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). A CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. See, e.g., Chothia et al., Nature 342:877-883, 1989. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008.

The term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. In contrast to polyclonal antibodies, which typically include different antibodies directed against different determinants (epitopes), each monoclonal antibody is directed against a single determinant on the antigen.

The term “humanized” antibody refers to forms of non-human (e.g., murine) antibodies that are chimeric immunoglobulins, immunoglobulin chains, or fragments thereof (such as Fv, Fab, Fab′, F(ab′)2 or other antigen binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) that are altered with respect to the original antibody, which are also termed one or more CDRs “derived from” one or more CDRs from the original antibody.

The term “dosage regime” refers to drug administration regarding formulation, route of administration, drug dose, dosing interval and treatment duration.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control.

Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

The term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable salt”, as used herein, refers to derivatives of the compounds defined herein, wherein the parent compound is modified by making acid or base salts thereof. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

The term “prodrug”, as used herein, refers to a pharmacological substance (drug) that is administered in an inactive (or significantly less active) form. Once administered, the prodrug is metabolized in the body (in vivo) into the active compound.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, compositions including one or more inhibitors of cancer cells may inhibit or reduce the activity and/or quantity of cancer cells by about 10%, 20%, 30%, 40%, 50%, 75%, 85%, 90%, 95%, or 99% from the activity and/or quantity of the same cells in equivalent tumor tissues of subjects that did not receive the inhibitor compositions.

The term “treating” or “preventing” a disease, disorder, or condition includes ameliorating at least one symptom of the disease or condition. Desirable effects of treatment include decreasing the rate of disease progression, ameliorating, or palliating the disease state, and remission or improved prognosis. For example, an individual is successfully “treated” if one or more symptoms associated with cancer are mitigated or eliminated, including, but are not limited to, reducing the proliferation of cancerous cells, decreasing symptoms resulting from the disease, increasing the quality of life of those suffering from the disease, decreasing the dose of other medications required to treat the disease, delaying the progression of the disease, and/or prolonging survival of individuals.

The term “biodegradable”, generally refers to a material that will degrade or erode under physiologic conditions to smaller units or chemical species that are capable of being metabolized, eliminated, or excreted by the subject. The degradation time is a function of composition and morphology.

The term “targeting moiety” means a moiety that localizes to or away from a specific locale. The moiety may be, for example, a protein, nucleic acid, nucleic acid analog, carbohydrate, or small molecule. The locale may be a tissue, a particular cell type, or a subcellular compartment. In some embodiments, the targeting moiety directs the localization of an active agent such as a microtubule polymerization inhibitor. In a particular embodiment, the targeting moiety is an antibody that binds specifically to the target cancer cells or the tumor region.

The term “prolonged residence time” means an increase in the time required for an agent to be cleared from a patient's body, or organ or tissue of that patient.

### II. Compositions

The combination therapies include administration of an effective amount of at least two active agents, one being a microtubule targeting agent or microtubule polymerization inhibitor (jointly referred to here as microtubule polymerization inhibitors unless otherwise specified) and the other being a polo-like kinase inhibitor, to a subject in need thereof.

A. Active Agents
1. Microtubule Targeting Agents or Microtubule Polymerization Inhibitors (Referred to Jointly as Microtubule Polymerization Inhibitors)

Microtubules are protein biopolymers formed through polymerization of heterodimers of α- and β-tubulins. Disruption of microtubules can induce cell cycle arrest in G2-M phase and formation of abnormal mitotic spindles.

Their importance in mitosis and cell division makes microtubules an attractive target for anticancer drug discovery. A number of naturally occurring compounds such as paclitaxel, epothilones, vinblastine, combretastatin, and colchicines exert their effect by changing dynamics of tubulin such as polymerization and depolymerization rates.

Microtubule targeting agents (MTA) are also named antimitotic agents that perturb not only mitosis but also arrest cells during interphase. MTAs are known to interact with tubulin through at least four binding sites: the laulimalide, taxane/epothilone, vinca alkaloid, and colchicine sites. Similar to paclitaxel, laulimalide can promote the tubulin-microtubule assembly, but binds to a different site on the microtubules (Pryor DE, et al., Biochemistry. 2002; 41:9109-15). Taxanes, including paclitaxel and docetaxel, bind to polymerized microtubules at the inner surface of the 3 subunit, and are widely used in the treatment of lung, breast, ovarian and bladder cancers. Taxanes promote tubulin stabilization, thereby interfering with tubulin dynamics. Vinca alkaloids, including vinblastine, vincristine, and vinorelbine, promote depolymerization of microtubules. They generally bind with high affinity to one or a few tubulin molecules at the tip of microtubules but do not copolymerize into microtubules. Indeed, vinblastine prevents self-association of tubulin by interacting at the interface between two αβ-tubulin heterodimers (Gigant B, et al., Nature. 2005; 435:519-22). The fourth group of microtubule interfering agents is represented by colchicine, which also induces microtubule depolymerization. In contrast to agents binding to the other three sites, colchicine binds with high affinity to tubulin that can become copolymerized into microtubules. Colchicine binding to β-tubulin results in curved tubulin dimer and prevents it from adopting a straight structure, due to a steric clash between colchicine and α-tubulin, which inhibits microtubule assembly (Ravelli RB, et al., Nature. 2004; 428:198-202). Microtubule targeting agents are reviewed for use in treatment of cancer in Čermák, et al. Eur.J.Cell Biol. 99(4):151075 (2020).

In some embodiments, the one or more microtubule targeting agents are capable of binding to one or more of laulimalide, taxane/epothilone, vinca alkaloid, and colchicine sites on tubulin. In some embodiments, the one or more microtubule targeting agents are taxanes including paclitaxel and docetaxel, although are more accurately referred to as microtubule depolymerizers or microtubule modulating or disrupting agents. In other embodiments, the one or more microtubule targeting agents are laulimalide, or derivatives and analogues thereof. In some embodiments, the microtubule targeting agents inhibit microtubule de-polymerization such as paclitaxel.

In some embodiments, the one or more microtubule polymerization inhibitors are monomethyl auristatin E, and/or maytansinoids such as DM1 and DM4.

The MTH1 inhibitor, TH588, is a microtubule-modulating agent that reduced microtubule plus-end mobility, disrupted mitotic spindles, and prolonged mitosis in a concentration-dependent but MTH1-independent manner (Gul, N. et al., Sci Rep 9, 14667 (2019)). Thus, in one embodiment, the microtubule targeting agent is TH588, or a prodrug, analog, or derivative, or pharmaceutically acceptable salt thereof. Structure of TH588 is shown below.

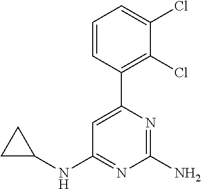 [SMILES: Nc1nc(NC2CC2)cc(-c2cccc(Cl)c2Cl)n1]

In some embodiments, the one or more microtubule polymerization inhibitors are one or more vinca alkaloids. Vinca alkaloids promote depolymerization of microtubules. Exemplary vinca alkaloids include vincristine, vinblastine, vinorelbine, vindesine, and vinflunine.

In a preferred embodiment, the microtubule polymerization inhibitor is vincristine, or a prodrug, analog, or derivative, or pharmaceutically acceptable salt thereof. The structure of vincristine is shown below.

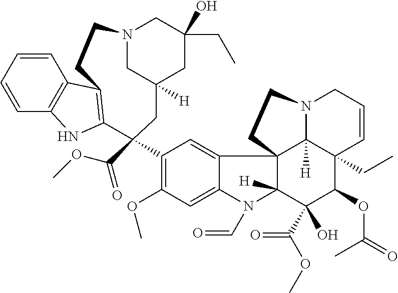

The main mechanism of vinca alkaloid cytotoxicity is due to their interactions with tubulin and disruption of microtubule function, particularly of microtubules comprising the mitotic spindle apparatus, directly causing metaphase arrest. The vinca alkaloids bind at sites on tubulin that are separate from those of the taxanes, colchicine, podophyllotoxin and guanosine-5′-triphosphate. Binding occurs rapidly and reversibly.

Targeted delivery can be used to reduce toxicity of one or more inca alkaloids to selectively accumulate at target cells or tissues. As discussed in more detail below, in some embodiments, the one or more vinca alkaloids are conjugated to a targeting moiety such as an antibody selective targeting to cancer cells or nearby tissues.

### Colchicine Binding Site Agents (CBSI)

Many of the CBSIs are based on natural products such as colchicinoids and combretastatins, as well as synthetic compounds such as ABT-751.

### Colchicine and ZD6126

A number of clinical trials have been done on colchicine for treatment of various diseases including cancer. However, the clinical use in treatment of cancer is hampered by its significant toxicity. ZD6126 is a water-soluble phosphate prodrug of N-acetylcolchinol structurally very similar to colchicine with potential anti-angiogenesis and antineoplastic activities (Goto H, et al., Cancer Res. 2002; 62:3711-5; and Lippert JW., Bioorg Med Chem. 2007; 15:605-15). ZD6126 was developed by AstraZeneca for the treatment of metastatic colorectal cancer. However, clinical trials were terminated due to apparent toxicity at pharmacological doses.

### CA-4 and its Analogs

Combretastatins are a class of stilbenoid phenols isolated from Combretum caffrum. Combretastatin A-4 (CA-4) is the most potent naturally occurring combretastatin known in regard to both tubulin binding ability and cytotoxicity. CA-4P (Zybrestat, fosbretabulin, and its salt fosbretabulin disodium, 3P) is the prodrug of CA-4 developed by OxiGene.

CA4P is a vascular disrupting agent (“VDA”)—in combination with Ipilimumab for the treatment of solid tumors with focus on melanoma in adult and pediatric melanoma. On May 4^{th} , 2020, FDA granted Rare Pediatric Disease Designation for CA4P/Fosbretabulin for the treatment of stage IIB-IV melanoma due to genetic mutations that disproportionately affect pediatric patients as a drug for a “rare pediatric disease.” Oxi4503 is a second generation VDA for the treatment of liquid tumors with focus on childhood leukemia. The FDA has designated OXi4503 (combretastatin Al-diphosphate; CA1P) for treatment of acute myeloid leukemia (AML) due to genetic mutations that disproportionately affect pediatric patients as a drug for a “rare pediatric disease.

An acute but transient increase in blood pressure is often the most clinically relevant toxicity associated with CA4P. Oxi4503 is combretastatin A-1 diphosphate (CA-1P) targeting tumor vasculature. It is a phosphorylated CA-4 analog developed by OxiGene for the treatment of solid tumors. A phase IB dose-escalating OX1222 study established the maximum tolerated dose for OXi4503 as a single agent or in combination with intermediate-dose cytarabine in patients with relapsed/refractory AML or myelodysplastic syndromes.

AVE8062 (ombrabulin) is another CA-4 analog that exerts its anticancer activity through disrupting blood vessel formation in tumors.

Compared with CA-4, it has improved water solubility and is orally available. AVE8062 has enhanced antitumor activity and decreased toxicity in a murine Colon-26 carcinoma model. It is also effective against a number of cancer cells that are resistant to taxanes (Kim TJ, et al., Cancer Res. 2007; 67:9337-45). In a phase I study, the combination of AVE8062 with docetaxel was well tolerated.

Phenstatin is also a CA-4 analog with the double bond of CA-4 being replaced by a carbonyl group. Phenstatin showed strong cytotoxicity and antitubulin activity similar to CA-4, but it is more stable compared with CA-4 that is known to be unstable in vivo due to the transformation from the active cis-configuration to the more stable but inactive trans-configuration. CC-5079 belongs to 1,1-diarylethene analogs of CA-4, which are called isocombretastatins A. CC-5079 is a dual inhibitor of tubulin polymerization and phosphodiesterase-4 (PDE4) activity. It showed antiangiogenic and antitumor activities. CC-5079 can arrest the cell cycle in G2/M phase, increase phosphorylation of G2/M checkpoint proteins, and induce apoptosis (Zhang LH, et al., Cancer Res. 2006; 66:951-9).

Podophyllotoxin, otherwise known as podofilox, is a non-alkaloid toxic lignan extracted from the roots and rhizomes of Podophyllum species. In 1890, Kiirsten isolated crystalline podophyllotoxin. Podophyllotoxin competitively inhibits the binding of colchicine. It binds to tubulin more rapidly than does colchicine. The utilization of podophyllotoxin as a lead in anticancer drug design has resulted in useful cancer fighting drugs such asetoposide, teniposide, and etoposide phosphate.

Steganacin, a lignan lactone from the alcoholic extract of Steganataenia araliacea Hochest, has significant anti-tumor activity in vivo against P388 leukemia in mice and in vitro against cells derived from a human carcinoma of the nasopharynx (KB). It was found that steganacin prevented the formation of the mitotic spindle that forms prior to the first cleavage. This suggested that steganacin, like other spindle poisons such as colchicine and podophyllotoxin, exerts its antimitotic activity through an effect on spindle microtubules (Kupchan SM, et al., J Am Chem Soc. 1973; 95:1335-6).

Nocodazole is a natural product that has been shown to have antimitotic and antitumor activity. The action of this agent is readily reversible and relatively rapid. Like podophyllotoxin and steganacin, this agent exerts its effect in cells by interfering with the polymerization of microtubules. However, the full therapeutic efficacy of this agent is limited owing to the development of various side effects in patients, including bone marrow suppression, neutropenia, leukopenia, and anemia (Attia SM. Journal of Applied Toxicology: JAT. 2011). Structure of nocodazole is shown below.

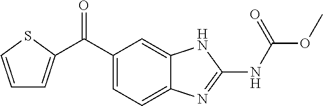 [SMILES: COC(=O)Nc1nc2ccc(C(=O)c3cccs3)cc2[nH]1]

Curacin A, originally purified as a major lipid component from a strain of the cyanobacterium Lyngbya majuscula isolated in Curaçao, is a potent inhibitor of cell growth and mitosis. It binds rapidly and tightly at the colchicine site of tubulin. A recurring structural theme in the colchicine binding site agents has been at least one and generally two aromatic domains (Hamel E. Medicinal Research Reviews. 1996; 16:207-31), while Curacin A, as a potent colchicine binding site antimitotic agent, is a major exception to this structural generalization in that it has no aromatic residue. Poor water-solubility and lack of chemical stability prevent the clinical development of curacin A, but synthetic analogs with improved bioavailability may provide new promise.

2-Methoxyestradiol (2-ME) is an endogenous estrogen metabolite, formed by hepatic cytochrome P450 2-hydroxylation of β-estradiol and 2-O-methylation via catechol O-methyltranseferase. This metabolite has attracted interest because of its potent inhibition of tumor vasculature and tumor cell growth. Because solid tumor growth is dependent on angiogenesis, the potent antiangiogenic activity and tubulin polymerization inhibition of 2-ME in vivo are of potential therapeutic value and have warranted further investigation in clinical trials. Some adverse effects of 2-ME included fatigue, nausea, diarrhea, neuropathy, edema, and dyspnea based on clinical trial data (Matei D, et al., Gynecol Oncol. 2009; 115:90-6). Studies have shown that 2-ME is metabolized by conjugation at positions 3 and 17 and oxidation at position 17. The conjugated forms of 2-ME are inactive, and oxidation to 2-methoxyestrone results in 10-to 100-fold loss in activity in vitro (LaVallee TM, et al., Mol Cancer Ther. 2008; 7:1472-82). In order to make metabolically stable analogs with improved anti-tubulin properties, ENMD-1198 was generated via chemical modification at 3 and 17 position. This agent also binds to the colchicine binding site in tubulin, induces G2/M cell cycle arrest and apoptosis, and reduces hypoxia-inducible factor (HIF)-1a levels. Studies also showed that ENMD-1198 was very potent at inhibiting endothelial cell proliferation, motility, migration, and morphogenesis. In addition, ENMD-1198 induced a significant decrease in vascular endothelial growth factor receptor (VEGFR)-2 protein expression in endothelial cells. Furthermore, ENMD-1198 is able to disrupt vascular structures very quickly.

ABT-751 (E7010) is an orally bioavailable tubulin-binding agent that was a phase II clinical trial for cancer treatment. It is a sulfonamide antimitotic that binds to the colchicine site on β-tubulin that leads to a block in the cell cycle at the G2/M phase, resulting in cellular apoptosis. ABT-751 was investigated in a phase I clinical trial to assess its PK profile and safety (Hande KR, et al., Clin Cancer Res. 2006; 12:2834-40). The maximum tolerated dose for the daily schedule was 250 mg/day. Dose-limiting toxicities included abdominal pain, constipation, and fatigue. ABT-751 was absorbed after oral administration with an overall mean Tmax of about 2 h. The PK properties of ABT-751 were dose-proportional and time independent. ABT-751 metabolism occurred primarily by glucuronidation and sulfation.

T138067 is an antimitotic agent (Shan B, et al., Proc Natl Acad Sci USA. 1999; 96:5686-91). This compound has been shown to covalently bind to Cys239 on β-tubulin isoforms 1, 2, and 4 by way of a nucleophilic aromatic substitution reaction. The covalent modification of β-tubulin prevents the polymerization of the a, β-tubulin dimers into microtubules. This leads to cell cycle arrest at the G2/M phase followed by apoptosis. T138067 is effective against a variety of tumors, including those that express the multidrug resistance (MDR) phenotype (IC50=11-165 nM). A phase II clinical trial showed that treatment with T138067 was tolerable with moderate hematologic and gastrointestinal toxicity. Neurotoxicity, an expected side effect, was minimal.

BNC-105P was developed by Bionomics (Australia) as a low-molecular-weight vascular disrupting agent (VDA) for treatment of cancers. BNC-105P is a phosphorylated prodrug that is rapidly transformed to the active form BNC-105 by nonspecific endogenous phosphatases in plasma and on endothelial cells (Patterson DM, et al., Drugs of the Future. 2007; 32:1025-32). BNC-105 exhibits selectivity (81-fold) for growth factor activated endothelial cells compared to quiescent human umbilical vein endothelial cells (HUVECs). A phase I study has been completed and the drug was shown to be generally well tolerated. A phase I/II study for BNC-105P in combination with everolimus were carried out to evaluate efficacy in patients with metastatic renal cell carcinoma (Sumanta Pal, et al., Clin Cancer Res. 2015 Aug. 1; 21(15): 3420-3427).

Indibulin (D-24851, ZIO-301) is an orally active anti-mitotic drug that is effective against various human tumor cell lines and xenografts, including taxane-resistant tumors. In preclinical studies indibulin lacks neurotoxicity that is largely associated with other tubulin binding drugs. The antitumor activity against MDR cancers, the lack of neurotoxicity, and the oral dosing make indibulin a promising candidate for further development as an anticancer drug. Though indibulin was reported not to overlap with the colchicine site, it was shown to partially compete for binding with “colchicine” site binders (40% inhibition). In vivo, oral application of indibulin showed a remarkable efficacy in the Yoshida AH13 rat sarcoma model without systemic toxicity being observed. Indibulin not only inhibits growth of tumor cell lines with different resistance phenotypes including MDR1 and multi-drug resistance-associated protein (MRP), but also retains its antitumor activity against cancer cell lines with resistance to cisplatin, the topoisomerase-I-inhibitor SN-38, and the thymidylate synthase inhibitors 5-FU and raltitrexed. Although indibulin also alters microtubule function, no neurotoxic effects on rats were seen at curative doses compared to paclitaxel and vincristine treatment groups.

EPC2407 (Crolibulin), MPI-0441138, and MPC-6827 (Azixa, Verubulin)

The 4-aryl-4H-chromenes, which were developed by EpiCept Corp. in California, inhibit tubulin polymerization, and induce apoptosis. Through structure-activity relationship (SAR) studies of the 4-aryl-4H-chromenes, the anticancer drug candidate EPC2407 with potent vascular disrupting activity and in vivo efficacy has been identified (Gourdeau H, et al., Mol Cancer Ther. 2004; 3:1375-84). MPI-0441138 is the lead compound for MPC-6827 discovered by EpiCept and identified as a highly active apoptosis inducer (EC50 for caspase activation of 2 nM) and as a potent inhibitor of cell proliferation (GI50 of 2 nM) in T47D cells (Sirisoma N, et al., J Med Chem. 2008; 51:4771-9). This compound inhibits tubulin polymerization and growth of Pgp overexpressing cells, and shows efficacy in the MX-1 human breast and PC-3 prostate cancer mouse models. A phase I study indicated that MPC-6827 was well tolerated at the recommended dose. The most common adverse events were nausea, fatigue, flushing, and hyperglycemia (Tsimberidou AM, et al., Mol Cancer Ther. 2010; 9:3410-9).

CYT997 was originally discovered as a structurally distinct, orally active microtubule targeting agent. It is now in phase II clinical trials for the treatment of selected cancers. CYT997 inhibits tubulin polymerization by binding at the colchicine binding site of tubulin. CYT997 blocks the cell cycle at the G2/M phase, and western blot analysis indicates an increase in phosphorylated Bcl-2, along with increased expression of cyclin B1 (Burns CJ, et al., Mol Cancer Ther. 2009; 8:3036-45). This compound also possesses favorable PK properties and is orally active in different tumor models, including paclitaxel-resistant cancer. CYT997 exhibits vascular disrupting activity in vitro by effects on the permeability of human umbilical vein endothelial cell monolayers, as well as in vivo on tumor blood flow.

MN-029 (denibulin) is a benzoimidazole carbamate that reversibly inhibits microtubule assembly, resulting in disruption of the cytoskeleton of tumor vascular endothelial cells. MN-029 was found to demonstrate striking antivascular effects in tumors, leading to the induction of necrosis and a consequential rapid loss of clonogenic neoplastic cells. This VDA also was successfully incorporated into conventional cisplatin or radiation therapy treatments (Shiand W, Siemann DW. Anticancer Res. 2005; 25:3899-904). One phase I clinical study of MN-029 in patients with advanced solid tumors showed that MN-029 was generally well tolerated and showed decrease in tumor vascular parameters (Ricart AD, et al., Cancer Chemotherapy and Pharmacology. 2011; 68:959-70). The most common toxicities of MN-029 included nausea, dose-related vomiting, diarrhea, fatigue, headache, and anorexia. No significant myelotoxicity, stomatitis or alopecia was observed in clinical.

CI-980 ((S)-(-)-NSC 613862) is one of a class of 1, 2-dihydropyrido[3, 4-b] pyrazines that inhibits tubulin polymerization presumably by interacting with the colchicine binding site of tubulin. The (R)-(+)-isomer NSC 613863 showed potency in several biological assays. However, the S-isomer is the more potent inhibitor on tubulin polymerization and cell proliferation (de Ines C, et al., Cancer Res. 1994; 54:75-84). CI-980 treated cells accumulate in the M-phase of the cell cycle and subsequently die. In sensitive tumor models, the potency for this agent is similar to that of vincristine, but the spectrum of antitumor activity is wider. CI-980 shows activity against a variety of cancer cells in vitro, including leukemia, melanoma, sarcoma, mammary adenocarcinoma, and colon adenocarcinomas.

CP248 and CP461 are derivatives of exisulind (Aptosyn, inhibitor of the enzyme cyclic guanosine monophosphate phosphodiesterase (cGMP-PDE)). Tubulin polymerization is believed to be their target. Both CP248 and CP461 cause growth inhibition and apoptosis in several cancer cell lines. There are at least two modes of inhibiting tumor cells identified for CP248. One is its inhibition of the cGMP-specific PDE2 and PDE5 and activating a protein kinase G mediated signaling pathway that triggers apoptosis. The other is its ability to bind to tubulin, inhibit its polymerization, and cause cells to be arrested in mitosis (Yoon JT, et al., Mol Cancer Ther. 2002; 1:393-404). CP461 is a member of a class of proapoptotic drugs that inhibit cyclic GMP phosphodiesterases specifically but not cyclooxygenase-1 or −2. It was in a phase I study for the treatment of patients with advanced melanoma. CP-461 inhibits the growth of a broad range of human tumor cell lines in vitro at micromolar concentrations. It selectively induces apoptosis in cancer cells but not normal cells (Sun W, et al., Clin Cancer Res. 2002; 8:3100-4).

TN16 is a tenuazonic acid derivative exhibiting anti-tumor effects in vitro and in vivo by inhibiting microtubule assembly and produces M phase arrest. TN16 has a structure distinct from the representative microtubule inhibitor colchicine, and yet it inhibits microtubule assembly, and prevents the stabilization of microtubules (Tripodi F, et al., J Med Chem. 2012; 55:2112-24).

### Maytansinoids

Maytansinoid binds to a site on tubulin. In some embodiments, the one or more microtubule polymerization inhibitors are one or more inhibitors binding to the maytansine site on tubulin. Exemplary maytansine site binders include maytansine, rhizoxin, and PM060104.

The structure of maytansine is shown below.

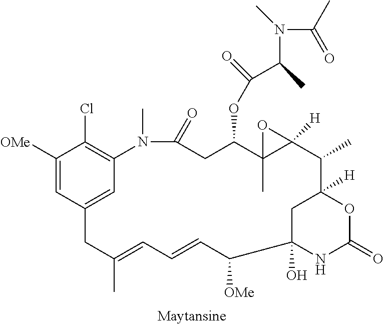 [SMILES: COc1cc2cc(c1Cl)N(C)C(=O)C[C@H](OC(=O)[C@H](C)N(C)C(C)=O)C1(C)O[C@H]1[C@H](C)[C@@H]1C[C@@](O)(NC(=O)O1)[C@H](OC)/C=C/C=C(\C)C2]

Antimitotic drugs include anti-tubulin agents such as maytansinoids known as DM1 and DM4. Structures of maytansinoids known as DM1 and DM4 are shown below.

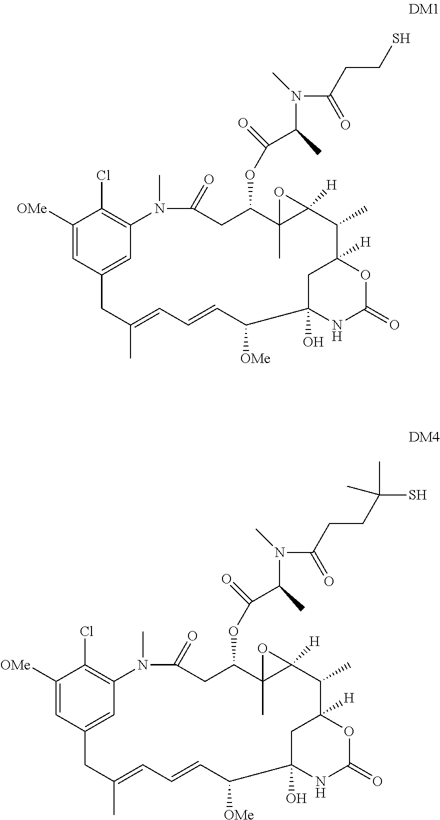 [SMILES: COc1cc2cc(c1Cl)N(C)C(=O)C[C@H](OC(=O)[C@H](C)N(C)C(=O)CCS)C1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCC(C)(C)S)C1(C)O[C@H]1[C@H](C)[C@@H]1C[C@@](O)(NC(=O)O1)[C@H](OC)/C=C/C=C(\C)C2]

### Antibody-Drug Conjugates

Antibody-drug conjugates (ADCs) are antibodies, or more typically antibody fragments, that bind surface proteins expressed specifically or preferentially on target cells (e.g., cancer cells), are internalized and then release their cytotoxic agent killing the target cells. There are three components to an ADC: the antibody or antibody fragment (jointly referred to here in as “antibody”), the cytotoxic agent and a chemical linker that connects them (FIG. 4). The antibody needs to bind to a surface epitope that will target the ADC to cancer cells. The linker needs to keep the cytotoxic agent attached to the antibody until internalization, and then release the cytotoxic agent. The cytotoxic agent needs to be exceptionally potent, since this system releases a limited amount of the drug inside cancer cells. Antibodies, cytotoxic agents, linkers, and methods for conjugation are described, for example, in U.S. Pat. No. 8,871,908.

Due to the low oral bioavailability of ADCs, ADCs are typically administered by intravenous injection. ADCs circulating in the blood bind their target cells. After binding, the ADC-antigen complex is internalized by clathrin-mediated endocytosis to form an early endosome containing an ADC-antigen complex. The early endosome eventually develops into a secondary endosome prior to fusion with the lysosome. For ADCs with cleavable linkers, the cleavage mechanism (e.g., hydrolysis, protease cleavage, disulfide bond cleavage) may occur either in the early endosome or in the secondary endosome, but not in lysosomal transport phase. However, for ADCs with non-cleavable linkers, the release of cytotoxic agents (drugs) is achieved by complete protein degradation in lysosomes: proton pumps in lysosomes create an acidic environment that promotes protease (e.g., cathepsin-B, plasmin) mediated proteolytic cleavage.

In preferred embodiments, one or more microtubule polymerization inhibitors are conjugated as cytotoxic agents on antibody-drug conjugates for selective targeting to cancer cells. Exemplary antimitotic antibody-drug conjugates include anti-tubulin agents such as monomethyl auristatin E and maytansinoids known as DM1 and DM4.

### Antibody and Target Antigen

The desirable properties of the ADC antibody portion include: 1) minimal immunogenicity; 2) high affinity and avidity for tumor antigen, and efficient internalization (ADC-target antigen complexes need to be internalized by receptor-mediated endocytosis, allowing them to release potent cytotoxic loads in cells); 3) longer circulating half-life.

In terms of specificity, an ideal target antigen needs to have two characteristics at the same time: 1) high expression on the surface of target cells; and 2) low expression in healthy tissues. In addition, the ideal shedding of the antibody should be as small as possible to prevent the free antigen from binding to the antibody in the circulation.

Typically, the antibody suitable for delivery of the active agents has binding specificity for one or more surface molecules associated with tumor cells such as CD33, CD30, HER2, CD22, CD79b, Nectin4, trophoblast cell surface antigen (TROP-2), BCMA, and CD19. In some embodiments, the antibodies for delivery of the cytotoxic agents are humanized to reduce immunogenicity in the subject to be treated. In some embodiments, one or more microtubule polymerization inhibitors are conjugated to one or more antibodies such as brentuximab, trastuzumab, polatuzumab, enfortumab, belantamab, tisotumab, gemtuzumab, inotuzumab, sacituzumab, and loncastuximab. In some embodiments, the antibodies for delivery of the cytotoxic agents are antibodies or fragments thereof that comprise a CDR that is at least 45%, at least 50%, at least 55%, at least 60%, at least 65%, at least 70%, at least 75%, at least 80%, at least 85%, at least 90%, at least 95%, or at least 99% identical to the amino acid sequence of a CDR of the above-listed clones and which exhibit immunospecific binding to the intended target. The determination of percent identity of two amino acid sequences can be determined by BLAST protein comparison.

### Cytotoxic Agents

The cytotoxic agent is the effector component of the ADC. In some embodiments, the cytotoxic agent of the ADC can target either DNA or tubulin.

The effect of tubulin inhibitors DM1, DM4, MMAE (auristatins monomethyl auristatin E) and MMAF (monomethyl auristatin F) is to inhibit microtubule polymerization, resulting in G2/M phase cell cycle arrest.

The basic parameters for selecting the cytoxic agent include conjugation, solubility, and stability. The structure of the cytotoxic agent should be such that it can be coupled to a linker. In addition, the water solubility of the toxic molecule and the long-term stability in the blood are important because the ADCs are prepared in an aqueous solution and administered intravenously.

In some embodiments, the cytotoxic agent is one or more microtubule polymerization inhibitors. Exemplary microtubule polymerization inhibitors are discussed above, including agents capable of binding to one or more of laulimalide, taxane/epothilone, vinca alkaloid, colchicine, and maytansine sites on tubulin. In preferred embodiments, the cytotoxic agent is MMAE (monomethyl auristatin E), MMAF (monomethyl auristatin F), maytansinoids such as DM1 and DM4, vinca alkaloids such as vincristine, vinblastine, vinorelbine, vindesine, and vinflunine, colchicinoids and combretastatins, TH588, or nocodazole.

The linker binds the cytotoxic agent to the mAb and maintains ADC stability in the systemic circulation. The chemical nature of the linker and the conjugating site play a crucial role in the stability, pharmacokinetic and pharmacodynamic properties of the ADC, as well as the therapeutic window. In some embodiments, the linker includes peptides and/or PEG chain. In preferred embodiments, the linker is designed to mitigate aggregation and immunogenicity of the ADC.

An ideal linker must have sufficient stability to ensure that the ADC molecules do not break apart early, can safely circulate through the bloodstream, and reach the target site. The linker must be able to break quickly during internalization to release the cytoxic agent. Linkers are classified into two types based on mechanism of cleavage: cleavable and non-cleavable. The former relies on physiological environment to release cytotoxic agents. A non-cleavable linker is a non-reducible bond with an amino acid residue in an mAb and is, therefore, more stable in the blood. An example of such a linker is a thioether linker, dependent on the lysosomal degradation of the mAb to release its cytotoxic agent.

The conjugating characteristics of the connector are critical to control the therapeutic window of the ADC. The drug to antibody ratio (DAR), or the amount of cytotoxic agent attached to the mAb, determines the potency and toxicity of the ADC. Although high drug loading can increase the potency of the ADC, it also increases off-target effects. To overcome the variability in the DARs of the ADC drugs in the production process, some studies have adopted site-specific conjugation to reduce variability, improve conjugating stability, and pharmacokinetic properties, and ultimately provide higher yield of the ADC drugs with a desired DAR. In some embodiments, the drug to antibody ratio (DAR) is between about 1:1 to about 20:1, inclusive; preferably between about 1:1 to about 10:1, inclusive; or between about 2:1 to about 5:1, inclusive.

Microtubule polymerization inhibitors used in ADCs include maytansinoids (DM1 and DM4) as well a derivative of auristatin E (MMAE). There are one DM1-based and three MMAE-based ADCs approved for the treatment of cancers and many more in various stages of clinical development. Because ADCs can deliver microtubule polymerization inhibitors specifically to cancer cells, the toxicity associated with the cytotoxic agent, such as neutropenia, can be dramatically reduced in frequency and severity. Table 1 lists FDA approved ADCs.

TABLE 1
FDA approved ADCs

| | | | | Cytotoxic agent/ | |
|---|---|---|---|---|---|
| | | | | Cytotoxic agent | Cytotoxic agent |
| ADC | Target | mAb | Linker | Class | Action |
| MYLOTARG ® | CD33 | IgG4 | acid | ozogamicin/ | DNA cleavage |
| (gemtuzumab | | | cleavable | calicheamicin | |
| ozogamicin) | | | | | |
| ADCETRIS ® | CD30 | IgG1 | enzyme | MMAE/ | microtubule |
| (brentuximab vedotin) | | | cleavable | auristatin | inhibitor |
| KADCYLA ® | HER2 | IgG1 | non- | DM1/ | microtubule |
| (adotrastuzumab | | | cleavable | maytansinoid | inhibitor |
| emtansine) | | | | | |
| BESPONSA ® | CD22 | IgG4 | acid | ozogamicin/ | DNA cleavage |
| (inotuzumab | | | cleavable | calicheamicin | |
| ozogamicin) | | | | | |
| POLIVY ® | CD79b | IgG1 | enzyme | MMAE | microtubule |
| (polatuzumab | | | cleavable | auristatin | inhibitor |
| vedotin-piiq) | | | | | |
| PADCEV ® | Nectin4 | IgG1 | enzyme | MMAE/ | microtubule |
| (enfortumab | | | cleavable | auristatin | inhibitor |
| vedotin-ejfv) | | | | | |
| ENHERTU ® | HER2 | IgG1 | enzyme | DXd/ | TOP1 inhibitor |
| (fam-trastuzumab | | | cleavable | camptothecin | |
| deruxtecan-nxki) | | | | | |
| TRODELVY ® | TROP2 | IgG1 | acid | SN-38/ | TOP1 inhibitor |
| (sacituzumab | | | cleavable | camptothecin | |
| govitecan-hziy) | | | | | |
| BLENREP ® | BCMA | IgG1 | non- | MMAF/ | microtubule |
| (belantamab | | | cleavable | auristatin | inhibitor |
| mafodotin-blmf) | | | | | |
| ZYNLONTA ® | CD19 | IgG1 | enzyme | SG3199/ | DNA cleavage |
| (loncastuximab | | | cleavable | PBD dimer | |
| tesirine-lpyl) | | | | | |
| TIVDAK ® | Tissue | IgG1 | enzyme | MMAE/ | microtubule |
| (tisotumab | Factor | | cleavable | auristatin | inhibitor |
| vedotin-tftv) | | | | | |

In the preferred embodiment, one or more microtubule polymerization inhibitors and one or more polo-like kinase (Plk) inhibitors are used as drug cytotoxic agents on antibody-drug conjugates (ADCs). In some embodiments, one or more microtubule polymerization inhibitors used as ADCs in the combination treatment with Plk inhibitors are one or more of ADCETRIS® (brentuximab vedotin), KADCYLA® (ado-trastuzumab emtansine), POLIVY® (polatuzumab vedotin-piiq), PADCEV® (enfortumab vedotin-ejfv), BLENREP® (belantamab mafodotin-blmf), TIVDAK® (tisotumab vedotin-tftv).

### 2. Polo-like Kinase Inhibitors

The combination therapies include one or more polo-like kinase (Plk) inhibitors. Polo-like kinases (Plks) are a family of conserved serine/threonine kinases involved in the regulation of cell cycle progression through G2 and mitosis. The catalytic domain of polo-like kinases is located in the N-terminus. The C-terminus of Plks contains one or two domains known as polo boxes that help localize the kinase to specific mitotic structures during mitosis. These include the centrosomes in early M phase, the spindle midzone in early and late anaphase and the midbody during cytokinesis.

Mammalian polo-like kinases include Plk1, Plk2/Snk, Plk3/Prk/FnK, Plk4/Sak, and Plk5. The polo-like kinase inhibitor can reduce or inhibit expression or activity of Plk1, Plk2/Snk, Plk3/Prk/FnK, Plk4/Sak, and Plk5.

For example, in some embodiments the inhibitor reduces or inhibits Plk1, Plk2/Snk, Plk3/Prk/FnK, Plk4/Sak, and/or Plk5 mRNA or protein expression. In some embodiments, the polo-like kinase inhibitor reduces or inhibits the kinase activity of Plk1, Plk2/Snk, Plk3/Prk/FnK, Plk4/Sak, and/or Plk5. In some embodiments, the polo-like kinase inhibitor reduces or inhibits protein interactions of more than one polo-like kinase, for example by targeting a conserved region of the proteins such as the polo box(es).

Plk1, named after the polo gene of Drosophila melanogaster, is a serine/threonine kinase that is crucial for the regulation of mitosis, and plays a key role in tumor cell proliferation. PLK1 expression is upregulated in a variety of tumor cell types and high expression is associated with increased aggressiveness and poor prognosis. In preferred embodiments, the combination therapies include one or more Plk1 inhibitors.

### Small Molecule PLK Inhibitors

In a preferred embodiment, the polo-like kinase inhibitor (Plk inhibitor) is a small molecule. “Small molecule” as used herein, refers to an organic molecule, inorganic molecule, or organometallic molecule having a molecular weight less than 2000, 1500, 1200, 1000, 750, or 500 atomic mass units. Polo-like kinase inhibitors are known in the art and include, for example, BI2536, volasertib (B1I6727), onvansertib, GSK461364, HMN-176, HMN-214, rigosertib (ON-01910), MLN0905, and Ro3280, several of which are discussed in Medema, et al., Clin. Cancer Res., 17:6459-6466 (2011).

Each of the Plk inhibitors, preferred dosages and routes of administration are discussed in more detail below, however, generally, the compounds can be administered to humans in an amount from about 0.0001 mg/kg of body weight to about 100 mg/kg of body weight per day. Generally, for intravenous injection or infusion, dosage may be lower than for other methods of delivery.

Some of the Plk inhibitors have been investigated for anti-cancer properties in preclinical experiments and clinical trials. In some embodiments, the dosage of Plk inhibitor used in combination therapies is the same as a dosage used to treat or prevent a cancer in a clinical trial, or a human equivalent to a dosage used to treat cancer in an animal study. Therefore, in some embodiments, the dosage is different than the dosage used to treat cancer. For example, the dosage can be lower than the dosage used to treat cancer, or the dosage can be higher than the dosage used to treat cancer provided that the dosage is safe and tolerable to the subject. Preferably, the dosage is at or below a maximum tolerated dose as determined in a clinical trial. In some embodiments, the maximum tolerated dose is 250 mg.

### Onvansertib

Onvansertib, also known as NMS1286937, NMS-P937, or PCM-075, is an orally bioavailable, small-molecule Polo-like kinase 1 (PLK1) inhibitor with potential antineoplastic activity. Upon administration, onvansertib selectively binds to and inhibits PLK1, which disrupts mitosis and induces selective G2/M cell-cycle arrest followed by apoptosis in PLK1-overexpressing tumor cells. Preclinical evaluation has shown high potency of the compound in proliferation assays, displaying low nanomolar activity on a large number of cell lines, representative of both solid and hematological tumors.

Ongoing clinical trials of onvansertib include a Phase 1b/2 study of onvansertib in combination with FOLFIRI and Bevacizumab for second line treatment of metastatic colorectal cancer in patients with a Kras mutation (see ClinicalTrials.gov Identifier: NCT03829410). The proposed treatment for Phase 1b is an escalating starting dose of onvansertib of 12 mg/m² orally on days 1 through 5 every 14-days over two treatment courses (1 cycle) in combination with FOLFIRI (180 mg/m² irinotecan, 400 g/m² leucovorin, 400 mg/m² bolus 5-fluorouracil (5-FU), and 2400 mg/m² continuous intravenous infusion 5-FU) and 5 mg/kg bevacizumab.

Treatment of cancer using Plk1 inhibitors such as onvansertib in combination with abiraterone has been described in U.S. Pat. No. 10,155,006. An ongoing clinical trial is a Phase 2 study of onvansertib in combination with abiraterone and prednisone in adult patients with metastatic castration-resistant prostate cancer (see ClinicalTrials.gov Identifier: NCT03414034). In this study, onvansertib is administered orally once daily (QD) at a dose of 24 mg/m² for 5 days (day 1 through day 5) out of a 14-day cycle or at a dose of 12 mg/m² for 14 days (day 1 through day 14) out of a 21-day cycle. In both regimens, beginning on day 1 and continuing uninterrupted throughout each cycle, patients also receive abiraterone and prednisone. In the same study, a third dosage combination including onvansertib administered orally once daily (QD) at a dose of 24 mg/m² for 5 days (day 1 through day 5) out of a 21-day cycle, and the same schedule for receiving abiraterone and prednisone as above, was also tested but discontinued. It is contemplated that any of the above clinical trial dosages and regimens can be used in the disclosed methods.

Thus, in preferred embodiments, the Plk inhibitor is onvansertib, or a prodrug, analog, or derivative, or pharmaceutically acceptable salt thereof. In some embodiments, the dosage of onvansertib can be in the range of 6 to 60 mg/m², inclusive. The structure of onvansertib is shown below.

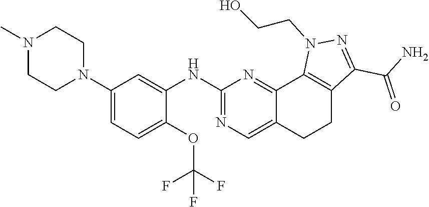 [SMILES: CN1CCN(c2ccc(OC(F)(F)F)c(Nc3ncc4c(n3)-c3c(c(C(N)=O)nn3CCO)CC4)c2)CC1]

### Dihydropteridinones

In some embodiments, the Plk inhibitor has the formula described in U.S. Pat. No. 6,806,272. The compounds have the structure:

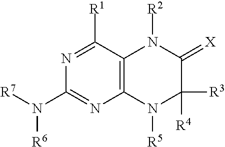

wherein

R¹ denotes a group selected from among hydrogen, NH₂, XH, halogen and a C₁-C₃-alkyl group optionally substituted by one or more halogen atoms, R₂ denotes a group selected from among hydrogen, CHO, XH, —X-C1-C2-alkyl and an optionally substituted C1-C3-alkyl group,

R³, R⁴ that may be identical or different denote a group selected from among optionally substituted C1-Cio-alkyl, C2-Cio-alkenyl, C2-Cio-alkynyl, aryl, heteroaryl, C3-C8-cycloalkyl, C3-C8-heterocycloalkyl, —X-aryl, —X— heteroaryl, —X-cycloalkyl, —X-heterocycloalkyl, —NR⁸-aryl, —NR⁸ -heteroaryl, —NR⁸-cycloalkyl and —NR⁸-heterocycloalkyl, or a group selected from among hydrogen, halogen, COXR⁸, CON(R⁸)₂, COR⁸ and XR⁸, or R³ and R⁴ together denote a 2-to 5-membered alkyl bridge that may contain 1 to 2 heteroatoms, R⁵ denotes hydrogen or a group selected from among optionally substituted C₁-C₁₀-alkyl, C2-Cio-alkenyl, C2-Cio-alkynyl, aryl, heteroaryl and —C₃-C₆-cycloalkyl, or R³ and R⁵ or R⁴ and R⁵ together denote a saturated or unsaturated C₃-C₄-alkyl bridge that may contain 1 to 2 heteroatoms, R⁶ denotes optionally substituted aryl or heteroaryl, R⁷ denotes hydrogen or CO—X—C₁-C₄-alkyl, and X in each case independently of one another denotes O or S, R⁸ in each case independently of one another denotes hydrogen or a group selected from among optionally substituted C₁-C₄-alkyl, C₂-C₄-alkenyl, C₂-C₄ alkynyl and phenyl, optionally in the form of the tautomers, the racemates, the enantiomers, the diastereomers and the mixtures thereof, and optionally the pharmacologically acceptable acid addition salts thereof.

Specific compounds of the formula above and other Plk inhibitors are described below.

BI2536

In a preferred embodiment, the Plk inhibitor is B1I2536, or a prodrug, analog, or derivative, or pharmaceutically acceptable salt thereof. BI2536 has the structure as shown below.

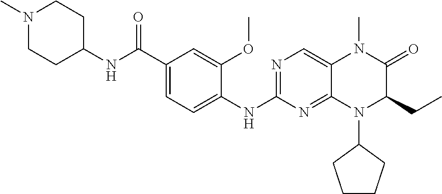 [SMILES: CC[C@@H]1C(=O)N(C)c2cnc(Nc3ccc(C(=O)NC4CCN(C)CC4)cc3OC)nc2N1C1CCCC1]

BI2536 is a potent Plk1 inhibitor with IC50 of 0.83 nM (Steegmaier, et al., Current Biology, 17:316-322 (2007)). It shows 4-and 11-fold greater selectivity for Plk1 against Plk2 and Plk3, respectively. In a preclinical experiment, BI2536 given i.v. once or twice per week was highly efficacious in diverse xenograft models with acceptable tolerability. The drug was believed to work by inhibiting cell proliferation through a mitotic arrest, and subsequently induction of tumor-cell death. Administration of BI2536 at 50 mg/kg once or twice per week significantly inhibited growth of HCT 116 xenografts with T/C of 15% and 0.3%, respectively. BI2536 treatment twice-weekly also lead to excellent inhibition of tumor-growth in BxPC-3 and A549 models with T/C of 5% and 14%, respectively (Steegmaier, et al., Current Biology, 17:316-322 (2007)).

BI2536 has been the subject of a number of clinical travels testing the safety and efficacy of the drug in a range of dosages and regimes and for treatment of a number of cancers. For example, in a randomized, open-label, phase I/II trial to investigate the maximum tolerated dose of the Polo-like kinase inhibitor BI2536 in elderly patients with refractory/relapsed acute myeloid leukemia, 68 elderly patients with relapsed/refractory AML were administered BI2536 on one of three schedules (day 1, days 1-3, and days 1+8). The maximum tolerated dose was 350 mg and 200 mg in the day 1 and days 1+8 schedules, respectively. The day 1-3 schedule appeared equivalent to the day 1 schedule and was discontinued early (Muller-Tidow, et al., Br. J. Haematol., 163(2):214-22 (2013)). Likewise, a phase I open-label dose-escalation study tested the maximum tolerated dose of intravenous BI2536 together with pemetrexed in previously treated patients with non-small-cell lung cancer. The patients received 500 mg/m² pemetrexed and escalating doses of BI2536 on day 1 every 3 weeks. Forty-one patients received BI2536 (100-325 mg). Two dose-limiting toxicities (DLT) occurred at BI2536 325 mg (grade 3 pruritus and rash; grade 4 neutropenia). Therefore, the maximum tolerated dose (MTD) for BI2536 in combination with pemetrexed was 300 mg (Ellis, et al., Clin. Lung Cancer, 14(1):19-27 (2013) Epub 2012 Jun 1). BI2536 at 200 mg combined with standard-dose pemetrexed was determined to have an acceptable safety profile. Other studies have suggested a lower MTD, e.g., 50-70 mg (Frost, et al., Curr. Oncology, 19(1):e25-35 (2012)).

An open, randomized, clinical phase II trial in patients with unresectable advanced pancreatic cancer was carried out to assess the efficacy, safety, and pharmacokinetics of BI 2536 administered in repeated 3-week cycles as a single i.v. dose of 200 mg on day 1 or as 60 mg doses on days 1, 2, and 3. Most common drug-related adverse events were neutropenia, leukopenia, fatigue, and nausea; most common grade 3/4-related events were neutropenia, leukopenia and thrombocytopenia

Therefore, in some embodiments, BI2536 is administered to a subject 1, 2, 3, or more times a week in a dosage of about 1-500 mg, preferably about 10-400 mg, more preferably about 50-300 mg, most preferably 60-250 mg. In a particular embodiment the dosage is 50, 100, 150, 200, 250, 300, or 350 mg of BI2536 administered to a subject once, twice, three times or more than three times a week, or once every two, three or four weeks. In some embodiments, BI2536 is administered by intravenous injection or infusion.

Volasertib (BI6727)

Like BI2536, BI6727 is an ATP-competitive kinase inhibitor from the dihydropteridinone class of compounds. BI6727 is a highly potent Plk1 inhibitor with IC50 of 0.87 nM. It also shows 6-and 65-fold greater selectivity to Plk1 relative to Plk2 and Plk3. BI6727 at concentrations up to M displays no inhibitory activity against a panel of >50 other kinases in vitro (Rudolph D, et al. Clin. Cancer Res., 15(9), 3094-3102 (2009)). BI6727 has the structure:

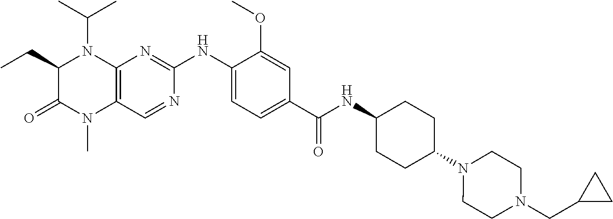 [SMILES: CC[C@@H]1C(=O)N(C)c2cnc(Nc3ccc(C(=O)N[C@H]4CC[C@H](N5CCN(CC6CC6)CC5)CC4)cc3OC)nc2N1C(C)C]

Preclinical experiments in a mouse model show that administration of BI6727 at −25 mg/kg/day significantly inhibits the growth of multiple human carcinoma xenografts including HCT116, NCI-H460, and taxane-resistant CXB1 colon carcinoma, accompanied by an increase in the mitotic index as well as an increase in apoptosis (Rudolph D, et al. Clin. Cancer Res., 15(9), 3094-3102 (2009)). Some in vivo studies indicate that BI6727 exhibits a better toxicity and pharmacokinetic profile than BI2536 (Harris, et al., BMC Cancer, 12, 80 (2012)).

BI6727 has been the subject of a number of clinical travels testing the safety and efficacy of the drug in a range of dosages and regimes and for treatment of a number of cancers. A phase I first-in-humans study of volasertib was conducted in 65 patients with advanced solid tumors, including 10 with non-small cell lung cancer (NSCLC). Volasertib was administered i.v. once every 3 weeks following a dose-escalation design (12-450 mg). The study reported neutropenia, thrombocytopenia, and febrile neutropenia as DLTs and an MTD of 400 mg (Gil, et al., J. Clin. Oncol., 28 Suppl 15:abstr 3061 (2010), Schoffski, et al., Eur. J. Cancer, 48(2):179-86 (2012)). 300 mg was the recommended dose for further development based on overall tolerability. In a phase I study of volasertib (BI 6727) combined with afatinib (BIBW 2992) in advanced solid tumors, the MTD was determined to be 300 mg of BI 6727, when administered in combination with afatinib (Peeters, et al., J. Clin. Oncol., 31 (suppl; abstr 2521) (2013)).

Therefore, in some embodiments, BI 6727 is administered to a subject 1, 2, 3, or more times a week in a dosage of between about 1-600 mg, preferably about 10-500 mg, more preferably about 50-400 mg, most preferably 100-350 mg. In a particular embodiment the dosage is 50, 100, 150, 200, 250, 300, 350, or 400 mg of BI 6727 administered to a subject once, twice, three times or more than three times a week, or once every two, three or four weeks. In some embodiments, BI 6727 is administered by intravenous injection or infusion.

Plogosertib (CYCI40)

Plogosertib (CYC140) is a selective, potent, and orally active ATP-competitive PLK1 inhibitor (IC50:3 nM). Plogosertib is an anti-cancer agent with anti-proliferative activity, and is in clinical testing for multiple types of cancer. Cyclacel is conducted human clinical trials of CYC140 in leukemias and solid tumors. Recent data suggest that PLK1 inhibition may be effective in KRAS-mutated metastatic colorectal cancer.

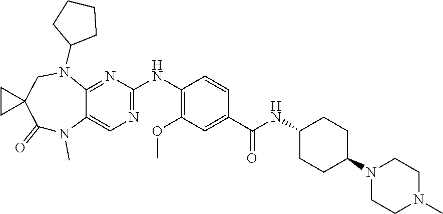 [SMILES: COc1cc(C(=O)N[C@H]2CC[C@H](N3CCN(C)CC3)CC2)ccc1Nc1ncc2c(n1)N(C1CCCC1)CC1(CC1)C(=O)N2C]

### Other Classes of Plk Inhibitors

The inhibitor may be a molecule other than a dihydropteridinones. Other classes of inhibitors include, but are not limited to, pyridopyrimidines (see U.S. Patent Application Publication No. 2010/004141 and WO 2009/112524), aminopyrimidines (see U.S. Patent Application Publication No. 2010/010014), substituted thiazolidinones (see European Patent Application No. EP 2141163), pteridine derivatives (see European Patent Application No. EP 2079743), dihydroimidazo[1,5-f]pteridines (see WO 2010/025073), metasubstituted thiazolidinones, (see U.S. Patent Application Publication No. 2010/048891), benzyl styryl sulfone analogues (see WO 2009/128805), and stilbene derivatives.

Specific inhibitors are discussed below:

GSK461364

GSK461364 inhibits purified Plk1 with Ki of 2.2 nM. It is more than 1000-fold selective for Plk1 against Plk2/3.

The structure for GSK461364 is

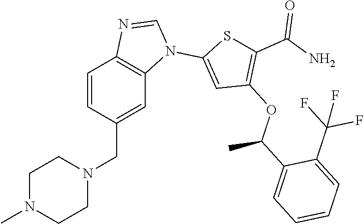 [SMILES: C[C@@H](Oc1cc(-n2cnc3ccc(CN4CCN(C)CC4)cc32)sc1C(N)=O)c1ccccc1C(F)(F)F]

Cell culture growth inhibition by GSK461364 can be cytostatic or cytotoxic but leads to tumor regression in xenograft tumor models under proper dose scheduling. In an animal model, dosages of 25, 50, and 100 mg/kg were administered via i.p. every 2 days or every 4 days (Gilmartin, et al., Cancer Res, 69(17), 6969-6977 (2009)).

A phase I first-in-humans study of GSK461364 was conducted in 27 patients with advanced solid tumors (Olmos, et al., Clin. Cancer Res., 17:3420-30 (2011)). The agent was administered i.v. following 2 schedules with different dosing (50-225 mg on days 1, 8, and 15 (schedule A) or 25-100 mg on days 1, 2, 8, 9, 15, and 16 (schedule B) on a 28-day cycle. DLTs included grade 4 neutropenia, sepsis, and pulmonary embolism. The final recommended phase II dose for GSK461364 was 225 mg administered intravenously in schedule A. Because of the high incidence (20%) of venous thrombotic emboli (VTE), coadministration of prophylactic anticoagulation agent is recommended.

Therefore, in some embodiments, GSK461364 is administered to a subject 1, 2, 3, or more times a week in a dosage of between about 1-400 mg, preferably about 10-350 mg, more preferably about 25-300 mg, most preferably 25-225 mg. In a particular embodiment the dosage is 50, 100, 150, 200, 250, 300, 350, or 400 mg of GSK461364 administered to a subject once, twice, three times or more than three times a week, or once every two, three or four weeks. In some embodiments, GSK461364 is administered by intravenous injection or infusion.

HMN-176 and HMN-214

HMN-176 is a stilbene derivative that is an active metabolite of the prodrug HMN-214. It does not directly inhibit the enzymatic activity of Plk1 but rather affects subcellular distribution of Plk1. The structures of HMN-176 and HMN-214 are (A) and (B) respectively:

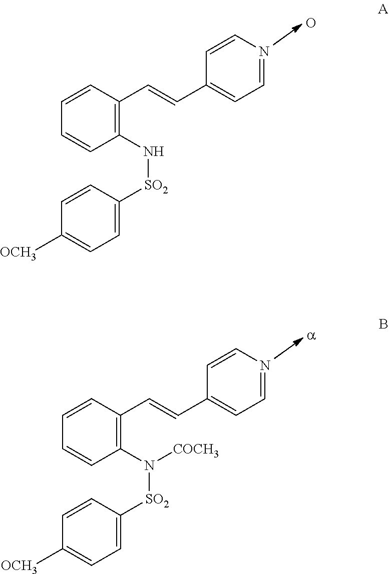

HMN-176 shows potent cytotoxicity toward various human tumor cell lines, and in mitotic cells, it causes cell cycle arrest at M phase through the destruction of spindle polar bodies, followed by the induction of DNA fragmentation. In preclinical experiments it was a potent antitumor activity in mouse xenograft models when administered at dosage of 10 mg/kg and 20 mg/kg on days 1 and 28 (Tanaka, et al., Cancer Res., 63:6942-6947 (2003).

A phase I pharmacokinetic study of HMN-214 in patients with advanced solid tumors, thirty-three patients were enrolled onto four dosing cohorts of HMN-214 from 3 to 9.9 mg/m²/d using a continuous 21-day dosing schedule every 28 days. A severe myalgia/bone pain syndrome and hyperglycemia were dose-limiting toxicities at 9.9 mg/m²/d, and the maximum tolerated dose and recommended dose on this schedule was determined to be 8.0 mg/m²/d (Garland, et al., Clin. Cancer Res., 1;12(17):5182-9 (2006)).

In another study, DLTs of prolonged neutropenia, febrile neutropenia, neutropenic sepsis, electrolyte disturbance, neuropathy, and myalgia were observed at doses of 24 to 48 mg/m² for 5 consecutive days every 4 weeks. MTD was established at the range of 18 to 30 mg/m², based on previous patient treatment load (Patnaik, J. Clin. Oncol., 22 Suppl:abstr 514.).

Therefore, in some embodiments, HMN-214 (or HMN-176) is administered to a subject 1, 2, 3, 4, 5, 6, or 7 times a week in a dosage of between about 1-100 mg/m², preferably about 2.5-50 mg/m², more preferably about 3-40 mg/m², most preferably 7.5-30 mg/m². In a particular embodiment the dosage is 1, 2, 3, 4, 5, 6, 7, 8, 9, or 10 mg/m² of HMN-214 (or HMN-176) administered to a subject once, twice, three times or more than three times a week, for example, on days 1-21 of a 28 day cycle. In another particular embodiment the dosage is 10 to 48 mg/m², preferably 18 to 30 mg/m² of HMN-214 (or HMN-176) administered once, twice, three times or more than three times a week, for example, days 1-5 of a 28 day cycle. In some embodiments, HMN-214 (or HMN-176) is administered orally.

Rigosertib (ON-01910)

The benzyl styryl sulfone analogue ON 01910 is an ATP-noncompetitive, multitargeted inhibitor of several tyrosine kinases and cyclin-dependent kinase 1 (Cdkl; IC50=18-260 nmol/L). It is reported to have a particularly strong potency (IC50=9-10 nmol/L) toward Plk1 (Gumireddy, et al., Cancer Cell, 7:275-86 (2005)). The structure of ON-01910 is shown below.

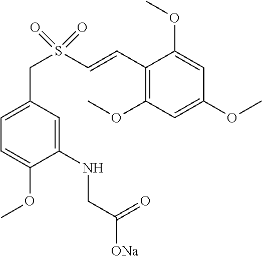

In preclinical animal studies in mouse xenograft models of Bel-7402, MCF-7, and MIA-PaCa cells, Rigosertib (250 mg/kg) inhibited tumor growth and (200 mg/kg) showed inhibition of tumor growth in a mouse xenograft model of BT20 cells (Gumireddy, et al., Cancer Cell, 7:275-86 (2005), Reddy, et al., J. Med. Chem., 54(18), 6254-6276 (2011)).

A phase I first-in-humans study of ON 01910 was conducted in 20 patients with advanced solid tumors (none with NSCLC). The agent was administered i.v. at 80 to 4,370 mg by accelerated titration design on days 1, 4, 8, 11, 15, and 18 in 28-day cycles (Jimeno, et al., J. Clin. Oncol., 26:5504-10 (2008). Grade 3 abdominal pain was reported as a DLT at an MTD of 3,120 mg.

In a clinical trial testing the safety and pharmacokinetics of oral ON 01910 in patients with myelodysplastic syndrome, ON 01910 was given twice a day up to 14 days at doses escalating from 70 mg to 700 mg.

Therefore, in some embodiments, ON 01910 is administered to a subject 1, 2, 3 or more days a week in a dosage of about 50-6,000 mg, preferably about 60-4,500 mg, more preferably about 150 mg-1,500 mg once daily, or 75-750 mg twice daily. In particular embodiments, ON 01910 is administered to a subject once, twice, three times or more than three times a week, or once every two, three or four weeks. In a specific embodiment, the drug is administered every day for 14 days. In some embodiments, ON 01910 is administered by intravenous injection or infusion.

MLN0905

MLN0905 is a potent inhibitor of PLK1 with IC50 of 2 nM. MLN0905 inhibits cell mitosis with EC50 of 9 nM and Cdc25C-T96 phosphorylation, a direct readout of PLK1 inhibition, with EC50 of 29 nM (Duffey, Med Chem, 55(1), 197-208 (2012)). The structure of MLN0905 is

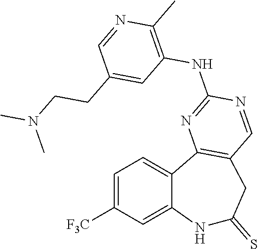 [SMILES: Cc1ncc(CCN(C)C)cc1Nc1ncc2c(n1)-c1ccc(C(F)(F)F)cc1NC(=S)C2]

Preclinical experiments indicate an effective dosage range of about 1 mg/kg-50 mg/kg. One study indicates a preferred dosage of about 3-15 mg/kg with a maximum tolerated dose on QD (daily) schedule to be 6.25 mg/kg and on the QDx3/wk (3-days on/4-days off) schedule to be 14.5 mg/kg (Shi, et al., Mol. Cancer Thera., 11(9), 2045-2053 (2012)).

RO3280

RO3280 is a potent, highly selective inhibitor of Polo-like kinase 1 (PLK1) with IC50 of 3 nM. The structure of RO3280 is shown below.

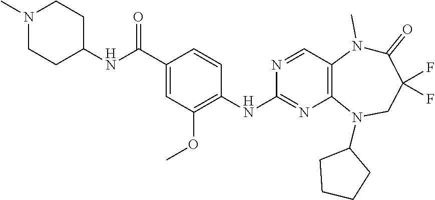 [SMILES: COc1cc(C(=O)NC2CCN(C)CC2)ccc1Nc1ncc2c(n1)N(C1CCCC1)CC(F)(F)C(=O)N2C]

RO3280 shows strong anti-proliferative activity against lung cancer cell line H82, colorectal cancer cell line HT-29, breast cancer cell line MDA-MB-468, prostate cancer cell line PC3 and skin cancer cell A375 with IC50s of 5, 10, 19, 12 and 70 nM, respectively. RO3280 also showed promising antitumor activity in nude mice implanted with HT-29 human colorectal tumors ranging from 72% tumor growth inhibition when dosed once weekly at 40 mg/kg, to complete tumor regression when dosed more frequently (Chen, et al., Bioorg. Med. Chem. Lett., 22(2), 1247-1250 (2012).

TAK-960

TAK-960 is an orally bioavailable, potent, and selective PLK1 inhibitor that has shown activity in several tumor cell lines, including those that express multidrug-resistant protein 1 (MDR1) (Hikichi, et al., Mol Cancer Ther. 11(3):700-9 (2012)). A Phase 1, open-label, dose-escalation study of orally administered TAK-960 has been completed.

CFI-400945 Fumarate

CFI-400945 is an inhibitor of polo-like kinase 4 (PLK4). Many tumors are shown to make too much PLK4. Phase 1 clinical trials of CFI-400945 fumarate delivered orally, at dose levels of 3, 6, 11, 16, 24, and 32 mg/day are currently underway (Mason, et al., Cancer Cell, V 26(2), pp.163-176(2014)).

### 3. Functional Nucleic Acid Inhibitors of PLK

In some embodiments, the polo-like kinase inhibitor is a functional nucleic acid that targets Plk1, Plk2/Snk, Plk3/Prk/FnK, Plk4/Sak, or Plk5. The functional nucleic acid can be, for example, an antisense molecule, aptamer, ribozyme, triplex forming oligonucleotide, external guide sequence, or RNAi that targets, inhibits, or reduces expression or translation of Plk1, Plk2/Snk, Plk3/Prk/FnK, Plk4/Sak, or Plk5 mRNA.

TKM-080301

In a particular embodiment, the functional nucleic acid inhibitor of PLK is TKM-080301. TKM-080301 is a lipid nanoparticle formulation of a small interfering RNA (siRNA) directed against PLK1 that has been shown to effect highly selective reductions in PLK1 mRNA in vitro and in tumor xenograft models in mice. TKM-080301 has been effective when given in a 30-minute intravenous infusion. Phase 1 and 2 clinical trials have been conducted, including doses ranging from 0.15 mg/kg per week to 0.9 mg/kg per week. Dose-limiting toxicities were observed at 0.9 mg/kg per-week.

Other suitable Plk (e.g., Plk1) inhibitors include, without limitation, SBE-13, ZK-Thiazolidinone, BI-4834 (a dihydropteridinone like BI2536 and volasertib), CAP-53194, Cyclapolin-9, DAP-81, GW843682X, MK-1496, PHA-680626, T521, UMB103, and UMB160.

In some embodiments, the Plk inhibitor does not inhibit the kinase activity of the Plk (e.g., Plk1), but rather, blocks its polo-box domain function. Exemplary polo-box domain blockers include Poloxin, Poloxin-2, Poloxime, Poloxipan, and Thymoquinone.

B. Formulations

Formulations and pharmaceutical compositions containing an effective amount of the compositions for reducing or inhibiting the activity of microtubule polymerization and Plk signaling, in a pharmaceutical carrier appropriate for administration to an individual in need thereof to treat one or more symptoms of cancer are provided.

In some embodiments, the pharmaceutical compositions can include one or more additional active agents. The pharmaceutical compositions can be formulated as a pharmaceutical dosage unit, referred to as a unit dosage form. Such formulations typically include an effective amount of a microtubule polymerization inhibitor or a Plk inhibitor, or a combination thereof with one or more other agents, which may also be used to treat symptoms associated with the toxicity of the cytotoxic agents, such as mucositis or nausea. In some embodiments the effective amount of microtubule polymerization inhibitor or Plk inhibitor in a combination therapy is different from that amount that would be effective for the microtubule polymerization inhibitor, or Plk inhibitor to achieve the same result individually. For example, in some embodiments the effective amount of microtubule polymerization inhibitor, or Plk inhibitor, is a lower dosage of the microtubule polymerization inhibitor, or Plk inhibitor in a combination therapy than the dosage of the microtubule polymerization inhibitor, or Plk inhibitor that is effective when one agent is administered without the other. Alternatively, in some embodiments the effective amount of microtubule polymerization inhibitor, or Plk inhibitor, is a higher dosage of the microtubule polymerization inhibitor, or Plk inhibitor in a combination therapy than the dosage of the microtubule polymerization inhibitor, or Plk inhibitor that is effective when one agent is administered without the other. In other embodiments, the dosage of one agent is higher and the dosage of the other agent is lower than one agent is administered without the other. In some case, the agents are not effective when administered alone, and only effective when administered in combination.

Typically, the formulation(s) are administered intravenously to deliver a safe and efficacious dosage. In certain embodiments, the compositions are administered locally, for example, by injection directly into a site to be treated (e.g., into a tumor). In some embodiments, the compositions are injected or otherwise administered directly into the vasculature onto vascular tissue at or adjacent to the intended site of treatment (e.g., adjacent to a tumor). Typically, local administration causes an increased localized concentration of the compositions that is greater than that which can be achieved by systemic administration. Targeting of the molecules or formulation can be used to achieve more selective delivery. In preferred embodiments, the microtubule polymerization inhibitor is targeted via conjugation to an antibody specifically targeting the cancer cells or tumor regions.

The active agents can be administered and taken up into the cells of a subject with or without the aid of a delivery vehicle. Concentrations of cytotoxic agent and volume can be adjusted as appropriate for the agent, vehicle and route of administration.

Appropriate delivery vehicles for the active agents are known in the art and can be selected to suit the particular inhibitor. For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Compositions can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art.

Oral formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, and coating compositions.

“Carrier” also includes all components of the coating composition that may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6” Edition, Ansel et.al., (Media, PA: Williams and Wilkins, 1995), which provides information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

See also Pharmaceutics and Pharmacy Practice, J. B. Lippincott Company, Philadelphia, Pa., Banker and Chalmers, eds., pages 238-250 (1982), and ASHP Handbook on Injectable Drugs, Trissel, 15th ed., pages 622-630 (2009).

### III. Methods of Use for Treatment of Cancer

Methods of using compositions for reducing or inhibiting the activity of microtubule polymerization and Plk signaling within cancer cells are provided.

Methods include administering to the subject an effective amount of one or more microtubule polymerization inhibitors and one or more Plk inhibitors in a subject in need thereof to reduce cancer cell proliferation and/or reduce cancer cell viability. These will typically not be administered together and will be administered using different routes of administration, such as oral and intravenous, daily with drug holidays versus once every three weeks.

It has been established that microtubule polymerization inhibitors can be used in combination with inhibitors of Plk to provide enhanced antitumor activity as compared to the use of either agent alone. Combination treatment of one or more microtubule polymerization inhibitors and one or more Plk inhibitors results in diminished cancer cell proliferation and reduction in tumor burden that is more than the additive effects of each class of inhibitors alone.

In preferred embodiments, the microtubule polymerization inhibitors and Plk inhibitors can be used in combination to provide enhanced antitumor activity as compared to the use of either agent alone. An effective amount of a microtubule polymerization inhibitor and one or more inhibitors of Plk are administered to the same patient, within the same treatment period, to decrease or inhibit the proliferation and/or viability of the cancer cells compared to untreated control cancer cells.

In preferred embodiments, one or more microtubule polymerization inhibitors are delivered as cytotoxic agents on antibody-drug conjugates. In some embodiments, one or more microtubule polymerization inhibitors and/or one or more inhibitors of Plk are delivered as cytotoxic agents on antibody-drug conjugates.

The microtubule polymerization inhibitor and Plk inhibitor can be administered locally (injection) and/or systemically (injection or orally) to the subject.

### Treatment Regimen

The combination therapies and treatment regimens are used for treatment of cancer or symptom thereof. Symptoms associated with cancer include overproliferation of transformed (cancer) cells, metastasis of cancer cells, pain, tiredness, swelling, and wasting. The two classes of cytotherapeutic agents, the microtubule polymerization inhibitor and the Plk inhibitor will be administered by a route based on the chemistry of the compound, its solubility and its uptake. For example, an antibody conjugate will typically be administered by injection. Other drugs may be formulated for oral administration. An effective dose is that which causes a decrease in one or more symptoms. Based on the studies to date, the effective dose of each class of compound will be less than if the compound is administered alone, since the combination shows “synergy”, i.e., the result of treating cancer cells with both classes of compounds is greater than what would be expected from treatment with either class alone, or the additive effective dose thereof. This may provide benefits to efficacy since a greater dose of the compounds in combination can be used that is safer than using the same amount of just one class of compound. The term “combination” or “combined” is used to refer to either concomitant, simultaneous, or sequential administration of the microtubule polymerization inhibitor and the Plk inhibitor. The combinations can be administered either separately but simultaneously (e.g., via separate intravenous lines into the same subject; one agent is given orally while the other agent is given by infusion or injection, etc.,), or sequentially (e.g., one agent is given first followed by the second). There may be a different time period between administered the two compounds, and there may be a different time period between treatment cycles.

When used for treating cancer, the amount of microtubule polymerization inhibitor present in a pharmaceutical dosage unit, or otherwise administered to a subject, will typically be the amount effective to reduce the proliferation, viability, or a combination thereof, of the cancer cells when administered in combination with a Plk inhibitor. In some embodiments the treatment regimen in which both classes of compounds are administered is that which is effective to reduce, slow or halt tumor progression, to reduce tumor burden, or a combination thereof. In some embodiments, the amount of the active agents is effective to alter a measurable biochemical or physiological marker. For example, if the cancer is prostate cancer, the amount of the active agents can be effective to reduce the level of prostate specific antigen (PSA) concentration in the blood compared to the PSA concentration prior to treatment.

In preferred embodiments, administration of the microtubule polymerization inhibitor and the Plk inhibitor achieves a result greater than when either the microtubule polymerization inhibitor and the Plk inhibitor are administered alone. For example, in some embodiments, the result achieved by the combination is partially or completely additive of the results achieved by the individual components alone. In the most preferred embodiments, the result achieved by the combination is more than additive of the results achieved by the individual components alone. In some embodiments, the effective amount of one or both agents used in combination is lower than the effective amount of each agent when administered separately. In some embodiments, the amount of one or both agents when used in the combination therapy is sub-therapeutic when used alone.

The effect of the combination therapy, or individual agents thereof can depend on the disease or condition to be treated or progression thereof. In some embodiments, the effect of the combination on a cancer can be compared to the effect of the individual agents alone on the cancer.

In some embodiments, the combination is improved over the individual components alone, for example, the cancer killing effect of the combination is similar to the individual components but the duration of efficacy of the treatment is longer.

A treatment regimen of the combination therapy can include one or multiple administrations of microtubule polymerization inhibitors, preferably as antibody-drug conjugates, preferably by injection. A treatment regimen of the combination therapy can include one or multiple administrations of Plk inhibitor, which may be by injection or orally. In certain embodiments, a microtubule polymerization inhibitor can be administered simultaneously with a Plk inhibitor.

In some embodiments, a microtubule polymerization inhibitor and a Plk inhibitor are administered sequentially, for example, in two or more different pharmaceutical compositions. In certain embodiments, the microtubule polymerization inhibitor is administered prior to the first administration of the Plk inhibitor. In other embodiments, the Plk inhibitor is administered prior to the first administration of the microtubule polymerization inhibitor. For example, the microtubule polymerization inhibitor and the Plk inhibitor can be administered to a subject on the same day. Alternatively, the microtubule polymerization inhibitor and the Plk inhibitor are administered to the subject on different days.

The Plk inhibitor can be administered at least 1, 2, 3, 5, 10, 15, 20, 24 or 30 hours or days prior to or after administering of the microtubule polymerization inhibitor. Alternatively, the Plk inhibitor can be administered at least 1, 2, 3, 5, 10, 15, 20, 24 or 30 hours or days prior to or after administering of the Plk inhibitor. In certain embodiments, additive or more than additive effects of the administration of microtubule polymerization inhibitor in combination with one or more Plk inhibitors is evident after one day, two days, three days, four days, five days, six days, one week, or more than one week following administration.

Dosage regimens or cycles of the agents can be completely or partially overlapping, or can be sequential. For example, in some embodiments, all such administration(s) of the microtubule polymerization inhibitor occur before or after administration of the Plk inhibitor.

Alternatively, administration of one or more doses of the microtubule polymerization inhibitor can be temporally staggered with the administration of Plk inhibitor to form a uniform or non-uniform course of treatment whereby one or more doses of microtubule polymerization inhibitor are administered, followed by one or more doses of Plk inhibitor, followed by one or more doses of microtubule polymerization inhibitor; or one or more doses of Plk inhibitor are administered, followed by one or more doses of microtubule polymerization inhibitor, followed by one or more doses of Plk inhibitor; etc., all according to whatever schedule is selected or desired by the researcher or clinician administering the therapy.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

It will be understood by those of ordinary skill that a dosing regimen can be any length of time sufficient to treat the condition in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

In some embodiments, the one or more doses of Plk inhibitor are administered orally in cycles, for example, administered daily for a period of time followed by a drug holiday. In some embodiments, one or more doses of microtubule targeting agents, preferably delivered as ADCs, are administered intravenously every 2-3 weeks. The therapeutic result of the compositions for reducing or inhibiting the activity of microtubule polymerization and Plk signaling can be compared to a control or to administration of either cytotoxic agent alone. Suitable controls are known in the art and include, for example, untreated cells or an untreated subject. A typical control is a comparison of a condition or symptom of a subject prior to and after administration of the active agents. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known in the art.

The combination therapies can be administered to a subject in combination with one or more adjunct therapies or procedures, or can be an adjunct therapy to one or more primary therapies or producers. The additional therapy or procedure can be simultaneous or sequential with the combination therapy. In some embodiments, the additional therapy is performed between drug cycles or during a drug holiday that is part of the combination therapy dosage regime. In preferred embodiments, the additional therapy is a conventional treatment for cancer, more preferably a conventional treatment for the particular cancer type, e.g., prostate or breast cancer. For example, in some embodiments, the additional therapy or procedure is surgery, a radiation therapy, or chemotherapy. For example, in a particular embodiment, combination therapies are used simultaneously or sequentially with a regime of a chemotherapeutic agent, e.g., docetaxel or cabazitaxel. In some embodiments, the adjunct or additional therapy is part of the combination therapy.

In some embodiments, the conventional cancer therapy is in the form of one or more additional active agents. Therefore, in some embodiments, the methods administer compositions in combination with one or more additional active agents. The combination therapies can include administration of the compositions for reducing/inhibiting the activity of microtubule polymerization and Plk signaling, and one or more additional active agents together in the same admixture, or in separate admixtures. Therefore, in some embodiments, the methods administer a pharmaceutical formulation including compositions for reducing/inhibiting the activity of microtubule polymerization and Plk signaling as well as one, two, three, or more additional active agents. Such formulations typically include an effective amount of compositions for reducing/inhibiting the activity of microtubule polymerization and Plk signaling, and an effective amount of an additional therapeutic, prophylactic or diagnostic agent. The additional active agent(s) can have the same, or different mechanisms of action. In some embodiments, the combination results in an additive effect on the treatment of the cancer. In some embodiments, the combinations result in a more than additive effect on the treatment of the disease or disorder.

The additional therapy or procedure can be simultaneous or sequential with the administration of the compositions for reducing/inhibiting the activity of microtubule polymerization and Plk signaling. In some embodiments the additional therapy is performed between drug cycles or during a drug holiday that is part of the composition dosage regime. For example, in some embodiments, the additional therapy or procedure is surgery, a radiation therapy, or chemotherapy.

Additional therapeutic agents include conventional cancer therapeutics such as chemotherapeutic agents, cytokines, chemokines, and radiation therapy, as discussed above. The majority of chemotherapeutic drugs can be divided into alkylating agents, antimetabolites, anthracyclines, plant alkaloids, topoisomerase inhibitors, and other antitumor agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In some embodiments, the additional therapy is a chemotherapeutic agent. Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5, and combinations thereof.

In the case of treating colorectal cancer, the additional chemotherapeutic therapy and regimens include FOLFOX (leucovorin calcium, fluorouracil, and oxaliplatin), CAPEOX (capecitabine and oxaliplatin), FOLFIRI (leucovorin calcium, fluorouracil, and irinotecan), FOLFOXIRI (leucovorin calcium, fluorouracil, oxaliplatin, and irinotecan), and 5-FU/LV (5-fluorouracil and leucovorin calcium), preferably as designated by the NCCN guidelines. An exemplary regimen of FOLFOX includes Day 1: Oxaliplatin 85 mg/m² IV over 2 hours, with Day 1: Leucovorin 400 mg/m² IV over 2 hours, followed by Days 1-2: Fluorouracil 400 mg/m² IV push on day 1, then 1,200 mg/m2/day x 2 days (total 2,400 mg/m² over 46-48 hours) IV continuous infusion; repeat cycle every 2 weeks. A further exemplary regimen of FOLFOX includes Day 1: Oxaliplatin 85 mg/m² IV over 2 hours, with Day 1: Leucovorin 400 mg/m² IV over 2 hours, followed by Days 1-2: Fluorouracil 1,200 mg/m²/day (total 2,400 mg/m² over 46-48 hours) IV continuous infusion; repeat every 2 weeks. In some embodiments, the additional chemotherapeutic therapy is FOLFOX plus bevacizumab; FOLFOX plus cetuximab; or FOLFOX plus panitumumab, preferably as designated by the NCCN guidelines.

In some embodiments, the compositions and methods are used prior to or in conjunction with an immunotherapy such as inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

In some embodiments, the additional therapy is adoptive T cell therapy. Methods of adoptive T cell therapy are known in the art and used in clinical practice. Generally adoptive T cell therapy involves the isolation and ex vivo expansion of tumor-specific T cells to achieve greater number of anti-tumor T cells than what could be obtained by vaccination alone. The tumor-specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol.,189(7):3299-310 (2012).

Historically, adoptive T cell therapy strategies have largely focused on the infusion of tumor antigen specific cytotoxic T lymphocytes (CTL) that can directly kill tumor cells. However, CD4+T helper (Th) cells such as Th1, Th2, Tfh, Treg, and Th17 can also be used. Th cells can activate antigen-specific effector cells and recruit cells of the innate immune system such as macrophages and dendritic cells to assist in antigen presentation by antigen presentation cells (APC), and antigen-primed Th cells can directly activate tumor antigen-specific CTL. As a result of activating APCs, antigen-specific Th1 have been implicated as the initiators of epitope or determinant spreading which is a broadening of immunity to other antigens in the tumor. The ability to elicit epitope spreading, broadens the immune response to many potential antigens in the tumor and can lead to more efficient tumor cell kill due to the ability to mount a heterogeneic response. In this way, adoptive T cell therapy can used to stimulate endogenous immunity. In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor-associated antigen. First generation CARs typically had the intracellular domain from the CD3 ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3ζ-CD28-41BB or CD3ζ-CD28-OX40, to further enhance effectiveness.

In some embodiments, the compositions and methods are used prior to or in conjunction with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen is delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis. In some embodiments, the compositions and methods are used to enhance the body's own anti-tumor immune functions.

Subjects to be Treated

In general, methods of administering combination therapies are useful in the context of treating cancer, including tumor therapy. All the methods described can include the step of identifying and selecting a subject in need of treatment, or a subject who would benefit from administration with the compositions.

In some embodiments, the cancers that are sensitive to additive and more than additive effects of the combination therapies are characterized by a specific gene or cytological profile. For example, cancer cells or tumors that are characterized by reduced expression or down-regulation of one or more genes or gene products involved in the mitotic spindle or mitotic spindle assembly can be more sensitive to the combination therapies than cancer cells that do not have reduced expression or down-regulation of one or more genes or gene products involved in the mitotic spindle or mitotic spindle assembly. Thus, in some embodiments, the methods involve the step of (a) analyzing the expression of one or more genes or gene products involved in mitosis, the mitotic spindle, mitotic spindle assembly in a sample from the subject, (b) selecting the subject for treatment if the sample is characterized by reduced expression or down-regulation of the one or more genes, and (c) administering to the selected subject an effective amount of a microtubule polymerization inhibitor in combination with an effective amount of a Plk inhibitor.

Typically, the subjects to be treated have a proliferative disease, such as a benign or malignant tumor. In some embodiments, the subjects to be treated have been diagnosed with stage I, stage II, stage III, or stage IV cancer.

The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods are useful for treating subjects having benign or malignant tumors by delaying or inhibiting the growth of a tumor in a subject, reducing the growth or size of the tumor, inhibiting, or reducing metastasis of the tumor, and/or inhibiting or reducing symptoms associated with tumor development or growth.

Malignant tumors that may be treated are classified according to the embryonic origin of the tissue from which the tumor is derived. Carcinomas are tumors arising from endodermal or ectodermal tissues such as skin or the epithelial lining of internal organs and glands. The compositions are particularly effective in treating carcinomas. Sarcomas, which arise less frequently, are derived from mesodermal connective tissues such as bone, fat, and cartilage. The leukemias and lymphomas are malignant tumors of hematopoietic ceils of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as colorectal cancer, peritoneal carcinomatosis, pancreatic cancer, multiple myeloma, sarcomas, brain, breast, esophageal, liver, lung, stomach, and uterine. In some embodiments, the compositions are used to treat multiple cancer types concurrently. The compositions can also be used to treat metastases or tumors at multiple locations. In some embodiments, the cancers to be treated are hepatocellular carcinoma, cholangiocarcinoma and medulloblastoma. In preferred embodiments, the cancers to be treated are prostate cancer, ovarian, breast, and bladder cancer.

Exemplary cancers that can be treated include brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipidus; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor); prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoid cystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or ureter); Wilms' tumor; bladder cancers including, but not limited to, transitional cell carcinoma, squamous cell cancer, adenocarcinoma, carcinosarcoma.

The methods and compositions as described are useful for both prophylactic and therapeutic treatment.

Therapeutic treatment involves administering to a subject a therapeutically effective amount of the compositions or pharmaceutically acceptable salts thereof as described after cancer is diagnosed.

In further embodiments, the compositions are used for prophylactic use i.e., prevention, delay in onset, diminution, eradication, or delay in exacerbation of signs or symptoms after onset, and prevention of relapse. For prophylactic use, a therapeutically effective amount of the compounds and compositions or pharmaceutically acceptable salts thereof as described are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms. Prophylactic administration can be used, for example, in the chemo-preventative treatment of subjects presenting precancerous lesions, those diagnosed with early-stage malignancies, and for subgroups with susceptibilities (e.g., family, racial, and/or occupational) to particular cancers.

In some embodiments, the cancer to be treated is prostate cancer. Plk1 expression is elevated in prostate cancer and shown to correlate with Gleason grade (Weichert W, et al., Prostate 60, 240-245(2004)). Prostate cancer is the most frequently diagnosed malignancy in men in Western countries. While localized prostate cancer can be effectively treated with surgery or radiation therapy, metastatic prostate cancer still remains incurable. For locally advanced or widespread disease, suppressing the tumor growth by hormone ablation therapy represents the common first therapeutic option (Beltran, et al., European Urology, 60:279-290 (2011)). Although initial therapy can lead to long-term remission, development of hormone ablation resistance can eventually occur, a standing referred to as castration-resistant prostate cancer (CRPC). Therefore, in some embodiments, the subject has a CRPC.

### IV. Kits

Medical kits are also disclosed. The medical kits can include, for example, a dosage supply of a Polo-like kinase 1 (Plk1) inhibitor and a microtubule polymerization inhibitor, or a combination thereof separately or together in the same admixture. In preferred embodiments, the microtubule polymerization inhibitors are formulated as antibody-drug conjugates having antibody specificity designed to target particular cancer types. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. The kits can include printed instructions for administering the compound in a use as described above.

The present invention will be further understood by reference to the following non-limiting examples.

### Examples

### Example 1: The Effect of Combination Treatment with Microtubule Polymerization Inhibitors and Polo-Like Kinase 1 (Plk1) Inhibitors is More than Additive

Material and Methods

Culturing of Cells

All cell lines were cultured in a humidified incubator at 37° C. and with 5% CO2, were maintained sub-confluent and used for no more than 20 passages. Media was supplemented with 10% fetal bovine serum (FBS), contained 2 mM glutamine and lacked antibiotics, unless otherwise noted. A549, BT-20, CCD-1112sk, CCD-18co, CFPAC1, HCT 116, HeLa, HT-29, HT55, MCF7, MDA-MB-157, MDA-MB-231, MDA-MB-415, MDA-MB-436, MDA-MB-453, MDA-MB-468, SW48, T98G, U-2 OS, U-87 MG, CAOV3, COV362, and JIMT1 cells were grown in DMEM; 22RV1, AU565, C₄-2, CAL33, COL0205,HCC38, HCT-15, KYSE150, LNCaP, LOVO, NCI-H1299, PC-3, ZR-75-1, HCC1419, HCC1569, HCC1954, ZR7530, OVCAR-4, OVCAR8 cells were grown in RPMI 1640; HT1197, HT1376, J82, UMUC3 cells were grown in MEM; OV90, TOV122D, TOV21G cells were grown in 1:1 MCDB105:Medial99 15% FBS; 59M, OAW28, OAW42 cells were grown in Advanced DMEM 0.023 IU/ml insulin; ES2, SKOV3 cells were grown in McCoy's 5A; JHOS2, JHOS4 cells were grown in 1:1 DMEM:HamF12; HUVEC cells were grown in EBM with EGM™-2 BULLETKIT™

Measurements of Drug Sensitivity and Greater than Additive Effects of Drug Combinations

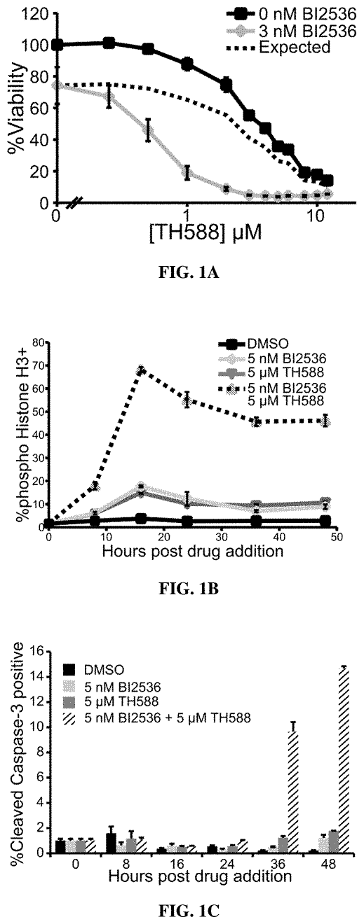

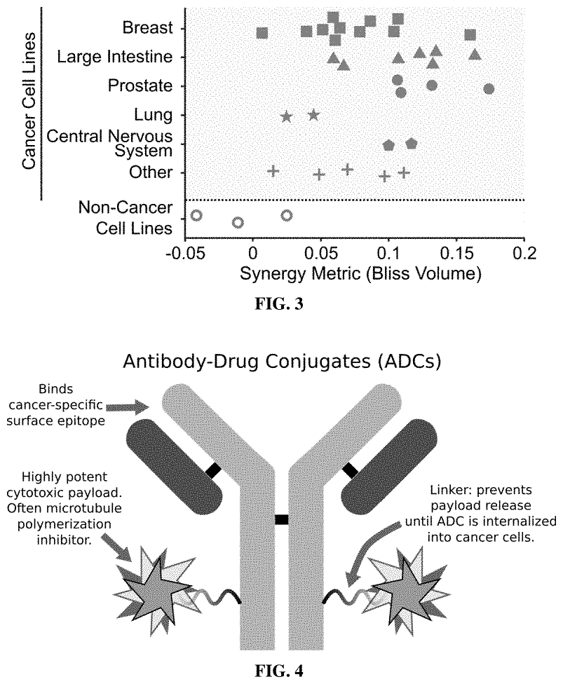

For dose response experiments and experiments that examine synergy as a measure of change in relative viability, cells were grown in 384-well plates in triplicate. The following day the cells were subjected to a drug dose matrix that consisted of increasing concentrations of a Plk inhibitor, increasing concentrations of a microtubule polymerization inhibitor, and all pairwise combinations. Drugs were dissolved in DMSO and diluted in the appropriate cell growth media while maintaining a constant final concentration of DMSO. After five days, relative viability was assessed using CELLTITER-GLO™ (Promega) Luminescent Cell Viability Assay according to the manufacturer's recommendations. Luminescence was measured using an INFINITE™ M200 Pro plate reader (Tecan Group Ltd.). Using these data, viability relative to DMSO control was calculated and compared to the expected additive response calculated according to the Bliss Independence model of drug additivity (Bliss. Annals of Applied Biology, 26(3): 585-615 (1939)). The dose response plots (for example FIG. 1A) show sensitivity to increasing concentrations of a microtubule polymerization inhibitor in the absence or presence of a constant amount of Plk inhibitor. Bars represent the standard error of the mean. The concentration of Plk inhibitor chosen to presented was based on a ˜20-30% reduction of viability when that drug was used at that dose in isolation. The entire amount of greater-than-additive response observed in the dose matrix is the integrated volume between the observed and expected surfaces generated by the response matrix and is also known as Bliss Volume. Bliss Volume synergy metrics are presented in FIG. 3 as the fractional change in volume between the expected and observed response surfaces.

Identification of Mitotic and Apoptotic Cells by Flow Cytometry

C₄-2 CRPC cells were plated in 6-well plates at a density of 300,000 cells per well and a total volume of 3 mls. The next day drugs were diluted in the appropriate growth media while maintaining a constant concentration of DMSO and added to the wells. After the indicated amount of time, cells were harvested by trypsinization. The media, trypsin and PBS wash were collected together to avoid loss of loosely attached or detached cells. Cells were fixed in 4% formaldehyde in PBS for 15 minutes, washed with PBS containing 1% bovine serum albumin (PBS-BSA), and then stored in methanol at −20° C. overnight. Cells were then washed twice in PBS-BSA 0.1% Tween-20, incubated with primary antibodies overnight at 4° C., washed with PBS-BSA 0.1% Tween-20 and incubated for 1 hour with fluorescent-dye conjugated secondary antibodies (diluted 1:200, Alexa Fluor, Molecular Probes) at room temperature for 1 hour. Primary antibodies included anti-phospho-serine 10 histone H3 (clone 3H10, Millipore) and anti-active caspase-3 (clone C₉₂-605, BD Pharmingen). The fixed cells were then washed with PBS-BSA 0.1% Tween-20 and resuspended in PBS containing 1 μg/ml 4,6-diamidino-2-phenylindole (DAPI, Molecular Probes) to stain DNA and analyzed using a BD™ LSRII flow cytometer (Becton Dickinson) and the FLOWJO™ software package. Shown is the average of three replicates±standard error of the mean.

### Animal Studies Using the Microwell Tumor-Implantable Device

C₄-2 CRPC xenograft tumors were grown in four to six week old castrated male NCR nude mice. Five million C₄-2 cells in 200 μl serum free media, mixed 1:1 with growth factor reduced MATRIGEL® (Invitrogen) were injected into the hind flank subcutaneously using a 23 gauge needle. Tumors took four to eight weeks to grow. Microdose drug delivery devices were manufactured, implanted, and analyzed as described by Jonas, O., et al. Science Translational Medicine, 7(284): 284ra57 (2015). Individual wells of these tumor implantable microdevices were loaded with the Plk inhibitor BI2536, the microtubule polymerization inhibitor TH588, or the combination of those drugs at half the dose relative to the wells containing the monotherapy. Tumors were excised 24 hours after device implantation, fixed in 10% formalin for 24 hours, and embedded in paraffin. Sections were stained with cleaved caspase-3 antibody (Cell Signaling Technologies, 9664) followed by detection with horseradish peroxidase conjugated secondary antibody and diaminobenzidine with hematoxylin used as a counterstain. Images were viewed using an EVOS® Cell Imaging System (Invitrogen) microscope, and scored using ImageJ in a blinded manner. Apoptotic index (Al) was calculated as the percentage of cells that were cleaved caspase-3-positive within 400 m of the reservoir-tissue interface.

All mouse studies were approved by the Massachusetts Institute of Technology Committee for Animal Care or Beth Israel Deaconess Institutional Animal Care and Use Committee, and conducted in compliance with the Animal Welfare Act Regulations and other federal statutes relating to animals and experiments involving animals and adheres to the principles set forth in the Guide for the Care and Use of Laboratory Animals, National Research Council, 1996 (Institutional Animal Welfare Assurance #A-3125-01).

Results

TH588 was developed as an inhibitor of an enzyme called MTH1 on the premise that this enzyme, which detoxifies a subset of oxidized nucleotides, was specifically required for cancer cell survival. Experimental data have shown that the effect of killing cancer cells by the combination of inhibitors of Polo-like kinase 1 (Plk1) with the TH588 drug was more than additive of each class of inhibitors alone (FIG. 1A). Plk1 is a kinase that is involved in entry into and progression through mitosis. Plk1 regulates many processes that are important to cell division, and has been of therapeutic interest for some time. Results indicate that the effect of the combination of TH588 with a Plk1 inhibitor including BI2536, BI6727 (volasertib), GSK461364, and onvansertib (PCM-075, NMS-1286937) is more than additive. This more than additive reduction in relative viability was accompanied by greater than additive mitotic arrest (FIG. 1B) and greater than additive apoptotic cancer cell killing (FIG. 1C) as judged by flow cytometry.

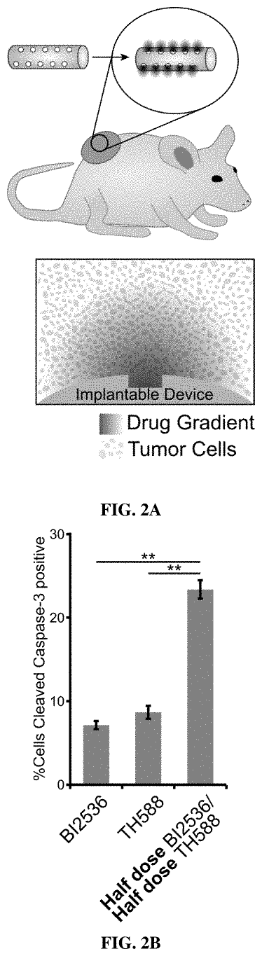

The more-than-additive effect on tumor cell killing was confirmed to occur in vivo using a tumor-implantable device for multiplexed drug delivery in C₄-2 CRPC xenograft tumors (FIGS. 2A-2B).

This combination of TH588 with a Plk1 inhibitor kills cancer cells of various origins and does not appear limited to a particular cancer type (FIG. 3). Approximately thirty cell lines of various origin were tested and the effect of tumor cell killing using the combination treatment was more than additive in the majority of these cell lines. Further data indicate that cancer cells with relatively low mRNA expression of components of the mitotic spindle are the ones that show the strongest synergistic responses. Importantly, the effect of killing by this drug combination in non-cancer cells was not more than additive as observed in majority of the cancer cell lines tested, and thus this combination of drugs targets a cancer-specific vulnerability (FIG. 3).

Further work was carried out to investigate the molecular basis of this more-than-additive efficacy of the Plk1 inhibitor TH588 combination treatment. It became clear that the effects of TH588 on cancer cells, and the cause of its enhanced efficacy when used with inhibitors of Plk1, were independent of its intended target, MTH1. In fact, knocking out MTH1 in cancer cells using CRISPR mutagenesis had no effect on viability, proliferation, sensitivity to TH588, or the enhanced efficacy from the combination of TH588 and Plk1 inhibitors. It was demonstrated that TH588 binds directly to tubulin and inhibits microtubule polymerization in vitro. The X-ray crystal structure of TH588 bound to tubulin was determined (PDB: 6QQN; Patterson JC et al., Cell Syst. 2019 Jul. 24; 9(1):74-92.e8) and the structure showed that the drug binds to the colchicine-binding site of (3-tubulin. Subsequent experiments confirmed that binding of TH588 to tubulin in cells is the dominant cause of its cytotoxic tumor killing ability, and of the more-than-additive killing effect that was observed when TH588 was combined with inhibitors of Plk1. Indeed, further study showed that other microtubule polymerization inhibitors such as nocodazole and vincristine also showed more-than-additive effect when used with Plk1 inhibitors in a manner similar to TH588 in killing tumor cells.

Therefore, it has been demonstrated that it is the specific inhibition of microtubule polymerization in combination with the Plk1 inhibitor that resulted in the enhanced killing cancer cells that was more than additive of each class of inhibitors alone. Microtubule polymerization inhibitors (most notably vincristine, vinblastine, and other vinka alkaloids) have been used for the treatment of various cancers for decades. While effective in some situations, their use can cause severe side effects, most notably peripheral neuropathy, and neutropenia. The neuropathy degrades patient quality of life, whereas the neutropenia renders patients susceptible to life-threatening infections. Plk1 inhibitors do not cause neuropathy, but some patients do develop neutropenia (although less frequently and usually not as severely as seen with microtubule poisons).

### Example 2: Antibody-Drug Conjugates (ADCs) of Microtubule Polymerization Inhibitors

Material and Methods

Culturing of Cells and Measurements of Drug Sensitivity and Greater than Additive Effects of Drug Combinations

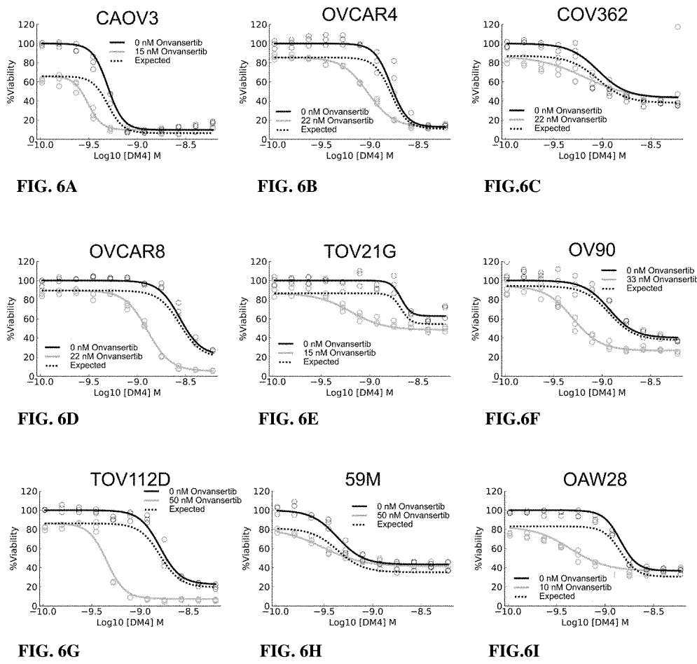

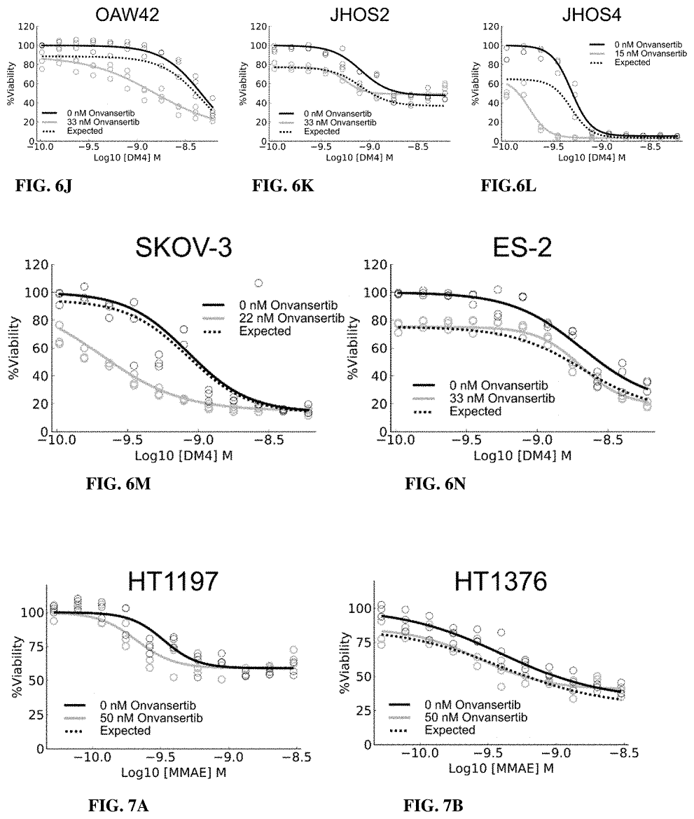

Cells were cultured and measurement of relative viability were performed as in Example 1. For FIGS. 6, 7 and 8 cells were subjected to dose matrices for 5 days and the response matrix was fit to sigmoid curves. Each row and column of the response matrix was fit to a sigmoid with the constraint that sigmoids from the rows and columns should be essentially equivalent where they intersect. As before, a particular dose of the Plk inhibitor was chose from this response matrix based on a 20-30% reduction in relative viability when used in isolation. Data points from the individual replicates are shown as circles.

Results

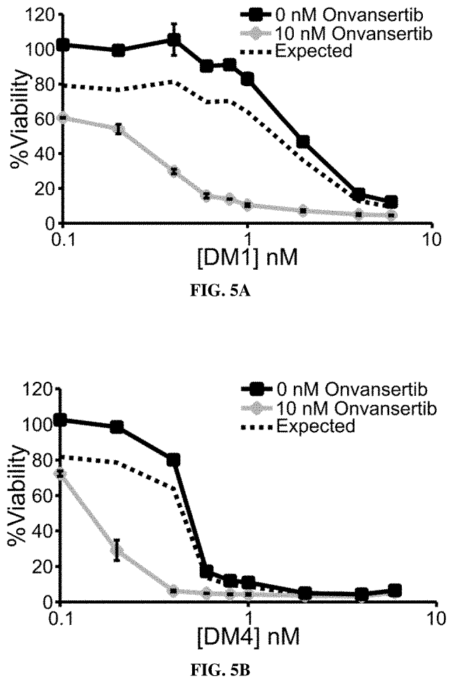

ADCs use antibodies directed against epitopes that are more highly abundant on the surface of cancer cells to deliver highly potent cytotoxic agents (FIG. 4), including microtubule polymerization inhibitors such as maytansinoids and MMAE. As with the other microtubule polymerization inhibitors tested, it has been established that the reduction in cancer cell viability is more than the additive reduction achieved by administering maytansinoids alone or Plk1 inhibitors alone as free agents (FIGS. 5A and 5B) in C₄-2 CPRC cells used in the Example above. This occurs at sub-nanomolar concentrations of DM1 and DM4, which is important since one limitation of ADCs is the ability to deliver large amounts of drug to cancer cells. The work with other microtubule polymerization inhibitors is representative of the effects of that class of drugs, and will apply to DM1, DM4 and MMAE, whether conjugated to antibodies or not. Plk1 inhibitors co-administered with microtubule-targeting ADCs should improve treatment of multiple cancers while avoiding life-threatening neutropenia.

Multiple microtubule-targeting ADCs have been approved for the treatment of cancer, and many more are at various stages of clinical development. Mirvetuximab soravtansine (Mirv-DM4) targets the folate receptor overexpressed in a subset of ovarian cancers and has completed a phase 3 clinical trial in this indication. DM4 is a maytansinoid derivative microtubule polymerization inhibitor. FIGS. 6A-6N present a panel of 14 ovarian cancer cell lines treated with the combination of combined Plk1 inhibitor (onvansertib) and DM4. Ten of fourteen of these cell lines had greater than additive responses to this drug combination, some of which responded very strongly to this combination.

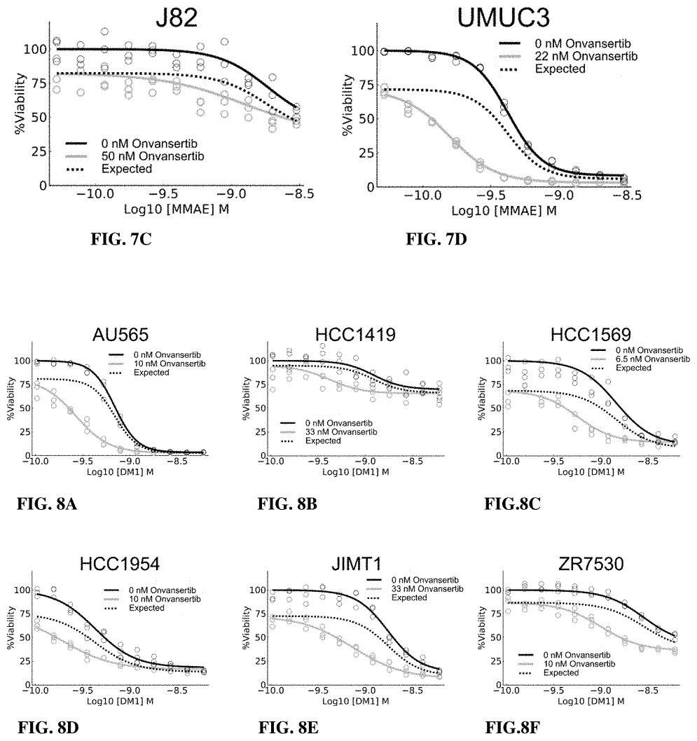

Enfortumab vedotin (PADCEV®) binds a protein nectin-4 that is particularly abundant on the cell surfaces of some bladder, breast, lung, colorectal and pancreatic cancers. Conjugated to this antibody is the microtubule polymerization inhibitor MMAE. Enfortumab vedotin was recently approved by the FDA for treatment of metastatic urothelial bladder cancer. Shown in FIGS. 7A-7D present a panel of four bladder cancer cell lines treated with the combination of Plk1 inhibitor (onvansertib) and MMAE. Varying amounts of greater than additive responses were observed with a very notable response to the combination in one of the four cell lines (UMUC3).

Trastuzumab emtansine (T-DM1) targets Her2 overexpressed in a subset of breast cancers and is FDA approved for treatment of this indication. DM1 is a maytansinoid derivative microtubule polymerization inhibitor. FIGS. 8A-8F show a panel of six Her2+ breast cancer cell lines the majority of which show a greater than additive response to combined Plk1 inhibitor (onvansertib) and DM1.

The current studies establish that Plk1 inhibition significantly enhances the cytotoxicity of microtubule poisons regardless of cancer type. An earlier study showed targeting PLK1 using a selective PLK1 inhibitor, volasertib, overcomes T-DM1 resistance via CDK1-dependent phosphorylation and inactivation of Bcl-2/xL in using mouse models specifically in the context of Her2+ trastuzumab-resistant breast cancer (Saatci O et al., Oncogene. 2018 April; 37(17):2251-2269).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

We claim:

1. Pharmaceutical compositions for use in treating cancer patients, the compositions comprising in combination an effective amount of
a microtubule polymerization inhibitor, and
a polo-like kinase (Plk) inhibitor,
wherein administration of the pharmaceutical compositions reduces cancer cell proliferation or viability, or one or more associated symptoms, to a greater degree or for longer duration than administering to the subject the same amount of microtubule polymerization inhibitor alone or the same amount of Plk inhibitor alone.

2. The pharmaceutical compositions of claim 1, wherein the reduction in cancer cell proliferation or viability in the subject with cancer is more than the additive reduction achieved by administering the microtubule polymerization inhibitor alone or the Plk inhibitor alone.

3. The pharmaceutical compositions of claim 1, wherein the microtubule polymerization inhibitor binds to a site on tubulin selected from the group consisting of laulimalide, taxane/epothilone, vinca alkaloid, and colchicine sites.

4. The pharmaceutical compositions of claim 3, wherein the microtubule polymerization inhibitor is a vinca alkaloid selected from the group consisting of vincristine, vinblastine, vinorelbine, vindesine, and vinflunine.

5. The pharmaceutical compositions of claim 4, wherein the microtubule polymerization inhibitor is vincristine, or a prodrug, analog, or derivative, or pharmaceutically acceptable salt thereof.

6. The pharmaceutical compositions of claim 3, wherein the microtubule polymerization inhibitor binds to colchicine-binding site on tubulin.

7. The pharmaceutical compositions of claim 6, wherein the microtubule polymerization inhibitor is selected from the group consisting of nocodazole, TH588, colchicinoids, combretastatins, ombrabulin, phenstatin, podophyllotoxin, steganacin, curacin A, 2-Methoxyestradiol, ABT-751, T138067, BNC-105P, indibulin, EPC2407, MPI-0441138, and MPC-6827, CYT997, MN-029, CI-980, CP248, CP461, and TN16.

8. The pharmaceutical compositions of claim 1, wherein the microtubule polymerization inhibitor is selected from the group consisting of monomethyl auristatin E, monomethyl auristatin F, and maytansinoids.

9. The pharmaceutical compositions of claim 8, wherein the microtubule polymerization inhibitor is a maytansinoid selected from the group consisting of DM1 and DM4.

10. The pharmaceutical compositions of claim 1, wherein one or more microtubule polymerization inhibitors are conjugated via a linker to an antibody or an antigen binding fragment thereof.

11. The pharmaceutical compositions of claim 10, wherein the antibody or an antigen binding fragment thereof specifically binds to a cell surface molecule highly expressed in a tumor cell compared to healthy cells.

12. The pharmaceutical compositions of claim 10, wherein the antibody or an antigen binding fragment thereof specifically binds to one or more cell surface molecules selected from the group consisting of CD33, CD30, HER2, CD22, CD79b, Nectin4, trophoblast cell surface antigen (TROP-2), BCMA, folate receptor alpha (FOLR1), and CD19.

13. The pharmaceutical compositions of claim10, wherein the microtubule polymerization inhibitors are conjugated via a linker to an antibody, or an antigen binding fragment thereof, selected from the group consisting of ADCETRIS® (brentuximab vedotin), KADCYLA® (ado-trastuzumab emtansine), POLIVY® (polatuzumab vedotin-piiq), PADCEV® (enfortumab vedotin-ejfv), BLENREP® (belantamab mafodotin-blmf), mirvetuximab soravtansine, and TIVDAK® (tisotumab vedotin-tftv).

14. The pharmaceutical compositions of claim 1, wherein the class of Plk inhibitors is selected from the group consisting of dihydropteridinones, pyridopyrimidines, aminopyrimidines, substituted thiazolidinones, pteridine derivatives, dihydroimidazo[1,5-f] pteridines, metasubstituted thiazolidinones, benzyl styryl sulfone analogues, stilbene derivatives, 4,5-dihydro-1H-pyrazolo[4,3-h]quinazoline derivatives, and combinations thereof.

15. The pharmaceutical compositions of claim 14, wherein the Plk inhibitor is selected from the group consisting of onvansertib, BI2536, volasertib (BI6727), GSK461364, HMN-176, HMN-214, rigosertib (ON-01910), MLN0905, TKM-080301, TAK-960, NMS-1286937, Ro3280, and CYC140.

16. The pharmaceutical compositions of claim 15, wherein the Plk1 inhibitor is onvansertib, or an analogue, derivative, or prodrug thereof.

17. A method for treating cancer in a subject in need thereof, comprising administering to a subject an effective amount of the pharmaceutical compositions comprising a microtubule polymerization inhibitor and a polo-like kinase (Plk) inhibitor, of claim 1.

18. The method of claim 17, wherein the pharmaceutical composition does not reduce or minimally reduces the proliferation and/or viability of healthy cells in the subject.

19. The method of claim 17, wherein the microtubule polymerization inhibitor and the polo-like kinase (Plk) inhibitor are administered via different routes and/or times within a treatment cycle.

20. The method of claim 19 wherein the microtubule polymerization inhibitor, and the polo-like kinase (Plk) inhibitor are administered either orally or by injection.

21. The method of claim 17, wherein the microtubule polymerization inhibitor or polo-like kinase (Plk) inhibitor, is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the other compound.

22. The method of claim 17, wherein the cancer is characterized by reduced expression or down-expression of one or more genes or gene products involved in the mitotic spindle or mitotic spindle assembly.

23. The method of claim 17, wherein the cancer is characterized by overexpression of Plk1.

24. The method of claim 17, wherein the cancer cells are insensitive to a microtubule polymerization inhibitor when the microtubule polymerization inhibitor is administered without co-administration of the Plk inhibitor.

25. The method of claim 17, wherein the cancer is selected from the group consisting of prostate cancer, breast cancer, ovarian cancer, colorectal cancer, pancreatic cancer, head and neck cancer, bladder cancer, and acute myeloid leukemia.

26. The method of claim 25, wherein the prostate cancer is castrate resistant prostate cancer.

27. The method of claim 17, wherein the subject is a human.

28. The method of claim 17 further comprising surgery or radiation therapy.

29. The method of claim 17 further comprising administering one or more immune checkpoint modulators selected from the group consisting of PD-1 antagonists, PD-1 ligand antagonists, and CTLA4 antagonists.

30. The method of claim 17further comprising adoptive T cell therapy, and/or a cancer vaccine.

31. A method for treating cancer in a subject in need thereof, comprising administering to a subject an effective amount of the combination of a microtubule polymerization inhibitor and a polo-like kinase (Plk) inhibitor, wherein administration of the pharmaceutical composition reduces cancer cell proliferation or reduces cancer cell viability, or reduces both cancer cell viability and proliferation in a subject with cancer, to a greater degree than administering to the subject the same amount of microtubule polymerization inhibitor alone or the same amount of Plk inhibitor alone,
wherein the microtubule polymerization inhibitor is conjugated via a linker to an antibody or an antigen binding fragment thereof, and
wherein the Plk inhibitor is onvansertib, or an analogue, derivative, or prodrug thereof.

32. The methods of 32, wherein the reduction in cancer cell proliferation or viability in the subject with cancer is more than the additive reduction achieved by administering the microtubule polymerization inhibitor alone or the Plk inhibitor alone.
